(19) United States
(12) Patent Application Publication
(10) Pub. No.: US 2010/0168012 A1
(43) Pub. Date: Jul. 1, 2010

(54) HUMAN CANCER THERAPY USING ENGINEERED MATRIX METALLOPROTEINASE-ACTIVATED ANTHRAX LETHAL TOXIN THAT TARGETS TUMOR VASCULATUTURE

(75) Inventors: Stephen H. Leppla, Bethesda, MD (US); Shihui Liu, Gaithersburg, MD (US); Thomas H. Bugge, Bethesda, MD (US); Brooke M. Curie, Washington, DC (US)

Correspondence Address: TOWNSEND AND TOWNSEND AND CREW, LLP, TWO EMBARCADERO CENTER, 8TH FLOOR, SAN FRANCISCO, CA, 94111, US

(73) Assignee: The Government of the United States of America as Represented by the Secretary of the Department of, Rockville, MD (US)

(21) Appl. No.: 12/519,362

(22) PCT Filed: Dec. 14, 2007
(86) PCT No.: PCT/US07/87664

§ 371 Date: Jan. 19, 2010

Related U.S. Application Data
(60) Provisional application No. 60/944,689, filed on Jun. 18, 2007.
(60) Provisional application No. 60/870,050, filed on Dec. 14, 2006.

(51) Int. Cl. A61K 38/16; A61P 35/00

(52) U.S. Cl. 514/12

(57) ABSTRACT

The present invention provides methods for inhibiting tumor associated angiogenesis by administering a mutant protective antigen protein comprising a matrix metalloproteinase-recognized cleavage site in place of the native protective antigen furin-recognized site in combination with a lethal factor polypeptide comprising a protective antigen binding site. Upon cleavage of the mutant protective antigen by a matrix metalloproteinase, the lethal factor polypeptide is translocated into cancer and endothelial cells and inhibits tumor associated angiogenesis.

### CROSS-REFERENCES TO RELATED APPLICATIONS

The present application claims priority to U.S. Ser. No. 60/870,050, filed Dec. 14, 2006, and U.S. Ser. No. 60/944,689, filed Jun. 18, 2007, each herein incorporated by reference in their entirety.

### STATEMENT AS TO RIGHTS TO INVENTIONS MADE UNDER FEDERALLY SPONSORED RESEARCH AND DEVELOPMENT

NOT APPLICABLE

### REFERENCE TO A “SEQUENCE LISTING,” A TABLE, OR A COMPUTER PROGRAM LISTING APPENDIX SUBMITTED ON A COMPACT DISK

NOT APPLICABLE

### BACKGROUND OF THE INVENTION

The majority of chemotherapeutic approaches to the treatment of cancer encompass agents that are directly cytotoxic to cancer cells. Such agents have typically exploited the unrestrained growth potential of cancer cells as compared to normal cells by targeting processes such as rapid cell division in cancer cells. Other therapeutic approaches are directed at inducing tumor cells to selectively undergo apoptosis or programmed cell death. Increasingly, another promising target for cancer treatment has been recognized—tumor associated angiogenesis. Tumor associated angiogenesis entails a complex interaction between tumor cells and endothelial cells in which new blood vessels are formed from pre-existing vessels, and involves the participation and interaction of a variety of cells and extracellular factors, such as endothelial cells, surrounding pericytes, smooth muscle cells, extracellular matrix (ECM), and angiogenic cytokines and growth factors (see, e.g., Rundhaug, Clinical Cancer Res., 9:551-554 (2003) for review).

It has increasingly been recognized that tumor angiogenesis is a necessary and required step for tumor development. In particular, the development of tumor vasculature is required for the establishment of a blood supply to and from a group of cancer cells that allows the transition from a small harmless cluster of cells to a large tumor. Angiogenesis is also required for the spread of a tumor, or metastasis. During metastasis, single cancer cells can break away from an established solid tumor, enter a blood vessel, and be carried to a distal site, where the escaped cell can implant and begin the growth of a secondary tumor. The vasculature surrounding a tumor would obviously play a key role in facilitating such a process. In fact, evidence now suggests that the blood vessels in a given solid tumor may in fact be mosaic vessels, comprised of endothelial cells and tumor cells. The mosaic nature of such vessels facilitates the ready and substantial shedding of tumor cells into the blood stream, allowing tumor cells to take residence at sites distant from the primary tumor. The subsequent growth of such metastases will, in turn, require a supply of nutrients and oxygen and a waste disposal pathway, provided by further tumor associated angiogenesis.

The recognition of the importance of tumor associated angiogenesis to the development and metastatic potential of various solid tumors has prompted a search for therapeutics that can block this process. Among the anti-angiogenesis based tumor therapies that have been explored include natural and synthetic angiogenesis inhibitors like angiostatin, endostatin and tumstatin, which are specific protein fragments derived from pre-existing structural proteins like collagen or plasminogen. The first FDA-approved therapy targeted at tumor associated angiogenesis is a monoclonal antibody directed against an isoform of VEGF, an angiogenic growth factor secreted by tumor cells that promotes blood vessel formation, and marketed under the name Avastin. This therapy has been approved for use in colorectal cancer in combination with established chemotherapy. While some anti-angiogenic agents are currently available, and research in this area continues, success to date has been limited. Accordingly, there is a need for additional and more effective agents that inhibit tumor associated angiogenesis. The present invention satisfies these and other needs.

### BRIEF SUMMARY OF THE INVENTION

Anthrax lethal toxin (LT) is selectively toxic to human melanomas with the BRAF V600E activating mutation due to its proteolytic activities toward the mitogen-activated protein kinase kinases. To decrease its in vivo toxicity, we generated a mutated LT that can only be activated by matrix metalloproteinases (MMPs). We found, surprisingly, that the MMP-activated LT has potent anti-tumor activity not only against human melanomas with the BRAF mutation, but also to a wide range of other tumor types, regardless of the BRAF status. This activity is largely due to the targeting of tumor angiogenesis. Moreover, the engineered toxin not only exhibits much lower toxicity than wild-type LT to mice, but also shows higher toxicity to tumors because of its greater bioavailability.

The majority of human melanomas, and a smaller fraction of other cancer types, contain a BRAF V600E mutation. These tumors have developed BRAF oncogene dependence and thus are sensitive to MEK inhibitors as well as to anthrax LT, as described herein and elsewhere. We show below that the MMP-activated LT has unanticipated broad and potent anti-tumor activity, exceeding wild-type LT, with respect to both safety and efficacy. The potent anti-tumor efficacy of the attenuated toxin is largely due to its inhibitory effects on tumor angiogenesis. Thus, our data shows that all tumor types would be responsive to the MMP-activated LT therapy as a result of inhibition of tumor associated angiogenesis as described herein. Furthermore, patients with tumors containing the BRAF mutation may derive additional benefits due to the direct toxicity of the toxin to the cancer cells.

In one aspect, the present invention provides a method of inhibiting tumor associated angiogenesis in a subject by (1) administering to the subject a therapeutically effective amount of a mutant PA protein comprising a matrix metalloproteinase 2-recognized cleavage site in place of the native PA furin-recognized cleavage site, wherein the mutant PA is cleaved by a matrix metalloproteinase; and (2) administering to the subject a therapeutically effective amount of an LF polypeptide comprising a PA binding site; wherein the LF polypeptide binds to cleaved PA and is translocated into a tumor associated endothelial cell, thereby inhibiting tumor angiogenesis. In some embodiments of this aspect, the mutant PA protein and the LF polypeptide are administered systemically to the subject.

In various embodiments of this aspect, the tumor can be a solid tumor. Examples of solid tumors include: lung cancer, colon cancer, melanoma, breast cancer, bladder cancer, thyroid cancer, liver cancer, pleural cancer, pancreatic cancer, ovarian cancer, cervical cancer, fibrosarcoma, neuroblastoma, and glioma.

In further embodiments of this aspect, the LF polypeptide can be native LF or else the LF polypeptide can be a fragment, such as LFn. Alternatively, the LF polypeptide can be a fusion protein.

In some embodiments, the matrix metalloproteinase 2 cleavage site has the sequence GPLGMLSQ. In some instances, the mutant PA is cleaved by a matrix metalloproteinase 2 from endothelial cells.

In further embodiments, the PA and LF, after translocation into a tumor associated endothelial cell, induces apoptosis of the endothelial cell. The endothelial cells in some embodiments may have an activated MAP kinase pathway. The translocated LF polypeptide and cleaved PA results in cleavage of MEKs1-4 and 6-7 in endothelial cells in some embodiments.

In another aspect, mutant PA is further cleaved by a matrix metalloproteinase 2 from a tumor cell. In such embodiments, the LF polypeptide binds to cleaved PA and is translocated into the tumor cell. In some embodiments, the translocated LF polypeptide and cleaved PA inhibit the expression of IL-8 mRNA in the tumor cell. In some embodiments, the tumor cells may have an activated MAP kinase pathway. An example of an activated MAP kinase pathway is one due to a BRAF V600E mutation. The translocated LF polypeptide and cleaved PA results in cleavage of MEK1, MEK3, and MEK4 in tumor cells in some embodiments.

### BRIEF DESCRIPTION OF THE DRAWINGS

FIG. 1 illustrates the cytotoxicity of the anthrax lethal toxins to human tumor cells. (A) Ten different NCI60 cell lines were incubated with various concentrations of PA or PA-L1 in the presence of 5 nM LF for 72 h, and the cell viability was measured as described in the Experimental Procedures section. Note that all the cells tested with the BRAF mutation were sensitive to the lethal toxins, whereas cells without the mutation (except MDA-MB-231 cells) were resistant to the toxins. (B) The same set of cell lines were also treated with PA or PA-L1 in the presence of 1.9 nM FP59 as described in (A). All the cells were sensitive to the toxins, demonstrating that the cells express MMP activities.

FIG. 2 illustrates that PA-L1/LF displays broad and potent anti-tumor activity regardless of the BRAF mutation status of the tumor. (A-C) Nude mice bearing human C32 melanoma (A), HT144 melanoma (B), or A549/ATCC lung carcinoma (C) were injected (i.p.) with 6 doses of PBS, PA/LF, or PA-L1/LF as indicated by red arrows (n=10 for each group). Weights of tumors in this and the following experiments are expressed as mean tumor weight±s.e.m. (D-E) PA-L1/LF causes extensive necrosis of A549/ATCC tumors. A549/ATCC tumor-bearing nude mice were treated with 4 doses of 30/10 μg of PA-L1/LF or PBS (at days 0, 2, 4, and 7). Two hours after injection of BrdU, tumors were dissected and subjected to histological analysis. H&E staining shows extensive toxin-dependent necrosis of a representative tumor treated with PA-L1/LF (D), which is observed in all the toxin-treated A549/ATCC tumors (E). (F-G) BrdU incorporation assay reveals remarkable DNA synthesis cessation in PA-L1/LF-treated but not PBS-treated A549/ATCC tumors. The tumor sections analyzed in (D-E) were stained with an antibody against BrdU 2 h after systemic administration of BrdU. Note, BrdU positive cells are easily detected in PBS-treated tumors, but hardly detected in viable areas of the toxin-treated tumors. (H) C57BL mice bearing mouse B16-BL6 melanomas or LL3 Lewis lung carcinomas were treated (i.p.) with 5 doses of PBS or PA-L1/LF as indicated (n=10 for each group). (I) PA-L1/LF displays much stronger anti-tumor activity than PA/LF. Nude mice bearing Colo205 colon carcinoma were treated (i.p.) with 6 doses of PBA, PA/LF, or PA-L1/LF as indicated (n=10 for each group). A significant difference (*, p<0.05; **, p<0.01) is shown between 15/5 μg of PA-L1/LF and 15/5 μg of PA/LF treated tumors. (J) PA-L1 has a longer plasma half-life than PA. Mice were injected (i.v.) with 100 μg of PA or PA-L1, euthanized at 2 h or 6 h, blood samples were collected, and PA protein concentrations were measured using ELISA. There is a significant difference (*, p<0.05; **, p<0.01) between PA and PA-L1. (K) C57BL/6 mice were injected i.p. with 6 doses of 5 or 15 μg of wild-type PA or PA-L1, respectively within a period of two weeks. Ten days later, the mice were bled, and the titers of the serum neutralizing antibodies against PA measured in a cytotoxicity assay using mouse macrophage RAW264.7 cells challenged with LT (75 ng/ml each of PA and LF). The titers of the PA neutralizing antibodies were expressed as mean of fold dilution±S.E. of the sera that could protect 50% of RAW264.7 cells from LT treatment. Note that the neutralizing activities from the mice treated with wild-type PA were approximately 6-fold higher that those from PA-L1 treated mice: PA vs. PA-L1 (6×5 μg): 1097±272 vs. 178±36, p=0.0002; PA vs. PA-L1 (6×30 μg): 1081±142 vs. 162±31, p=0.0004.

FIG. 3 illustrates the potent anti-tumor activity of PA-L1/LF is not solely dependent on its inhibitory effects on IL8. (A) Angiogenic factor profiling RT-PCR analysis reveals that the expression of IL8 by tumor cells is down-regulated by anthrax lethal toxin. Colo205, A549/ATCC, HT144, and HT29 cells were treated with or without PA/LF (10/3.3 nM) for 8 h, then the total RNA was isolated, and subjected to the angiogenic factor RT-PCR profiling analyses following the recommendations of the manufacturer. Note that IL8 is consistently down-regulated by PA/LF in all four cancer cell lines. ANGP1, angiopoietin 1; CSF3, colony stimulating factor 3; ECGF1, endothelial cell growth factor 1; FGF1 and FGF2, fibroblast growth factor 1 and 2; FST, follistatin; HGF, hepatocyte growth factor; LEP, leptin; PDGFB, platelet derived growth factor B; PGF, placental growth factor. (B-C) Both A549/ATCC carcinomas (B) and C32 melanomas (C) transfected with lethal LT ‘resistant’ IL8 retain susceptibility to PA-L1/LF. Nude mice bearing tumors transfected with IL8 or the empty vector were treated with 6 doses of 30/10 μg of PA-L1/LF or PBS. PA-L1/LF shows potent anti-tumor activity against the tumors transfected with either IL8 or the empty vector.

FIG. 4 illustrates that PA-L1/LF demonstrates potent anti-angiogenic activities. (A) Sections of A549/ATCC tumors treated with PBS or PA-L1/LF, as described in FIG. 2D, were stained with an antibody against the endothelial cell marker CD31. CD31-positive structures were quantified using the Northern Eclipse Image Analysis Software (Empix Imaging, North Tonawanda, N.Y.). In inserts, black arrows point to the examples of CD31-positive endothelial cells; dash line, the boundary between the tumor and its surrounding normal tissues. N, necrotic area; V, area with viable cancer cells. (B) Directed in vivo angiogenesis analysis demonstrates that PA-L1/LF can inhibit tumor cell independent in vivo angiogenesis. There is a significant difference (**, p<0.01) between the angioreactors treated with PBS (n=8) and treated with PA-L1/LF (15/5 ug, n=8; 30/10 ug, n=10). (C) Anthrax toxin receptors-deficient CHO tumors are susceptible to PA-L1/LF. CHO PR230 tumor-bearing nude mice were injected (i.p.) with 6 doses of 30/10 μg of PA-L1/LF as indicated (n=6 for each group). There is a significant difference (*, p<0.05) between the tumors treated with PA and PA-L1.

FIG. 5 illustrates that PA-L1/LF impairs the function of primary human endothelial cells. (A) PA protein-dependent translocation of LF into the cytosol of HMVEC and HUVEC cells. HUVEC and HMVEC cells were incubated with either PA-L1/LF (6 nM/6 nM) or PA/LF (6 nM/6 nM) for 2 or 4 h. The binding and proteolytic processing of PA proteins, the binding and translocation of LF, and the MEKs cleavages were detected by Western blotting using the corresponding antibodies. The non-specific bands, indicated by the arrow heads left of images, served as protein loading controls in these experiments. (B-C) Cytotoxicity of PA-L1/FP59 (B) and PA-L1/LF (C) to human primary vascular endothelial cells. HUVEC and HMVEC were treated with the indicated toxins as described in FIG. 1. The expression of MMPs by the endothelial cells was evidenced by their high sensitivity to PAL1/FP59. (D) PA-L1/LF can efficiently inhibit the migration of vascular endothelial cells toward angiogenic factors-containing endothelial cell growth medium (GM). The experiments were performed as described in the Experimental Procedures section. SFM, serum and angiogenic factors free medium.

FIG. 6 illustrates that PA-L1/LF delays, but does not prevent, incisional skin wound healing. (A) C57BL/6 mice with the incisional skin wounds were treated with either PA-L1/LF (30/10 ug) (n=7) or PBS (n=8) three times per week until all the wounds were healed. The average wound healing time was delayed for the toxin-treated mice compared to the mock-treated group (14.5 days vs. 10 days, p<0.001, Mann-Whitney U-test, two-tailed). (B) Representative examples of the appearance of skin wounds from mice treated with PA-L1/LF (left) or PBS (right) at days 5-9.

### DETAILED DESCRIPTION OF THE INVENTION

### I. Introduction

Tumor associated angiogenesis, as used herein, refers generally to the ability of a tumor cell to promote the formation of a vasculature to supply the tumor cell with nutrients and a means to remove metabolic waste products. Accordingly, tumor associated angiogenesis is a complex process by which new blood vessels are formed from existing vessels to provide a blood supply to tumor cells. Angiogenesis involves multiple interactions between endothelial cells, surrounding pericytes, smooth muscle cells, ECM, and angiogenic cytokines and growth factors. The multiple steps of angiogenesis include degradation of the basement membrane surrounding an existing vessel, migration and proliferation of endothelial cells into the new space, maturation, differentiation, and adherence of the endothelial cells to each other, and lumen formation. Angiogenesis can be initiated by the release of proangiogenic factors (e.g., VEGF, bFGF, TNF-α, IL-8, among others) from inflammatory cells, mast cells, macrophages, or tumor cells (see, e.g., Rundhaug, Clinical Cancer Res., 9:551-554 (2003) for review). These factors bind to their respective cell-surface receptors on endothelial cells, leading to the activation of these previously quiescent cells. Activation of quiescent endothelial cells results in the induction of cell proliferation, increased expression of cell adhesion molecules (e.g., integrins), secretion of MMPs, and increased migration and invasion. In particular, VEGF has been shown to be a potent mitogen and chemoattractant for endothelial cells and induces the release of MMP-2, MMP-9, and MT1-MMP by endothelial cells (see, e.g., Rundhaug, supra).

Thus, tumor associated angiogenesis involves a system of communication between tumor cells and preexisting endothelial cells that results in the formation of new blood vessel branches that supply nutrients to the tumor and that remove waste products from the tumor. In part, the process entails the release from tumor cells of proangiogenic factors such as VEGF, bFGF, IL-8, among others, as well as, the release of proteases such as MMPs to degrade the basement membrane surrounding tumor cells to facilitate the diffusion of proangiogenic factors to their corresponding cell surface receptors on endothelial cells. Upon the binding of tumor released proangiogenic factors to endothelial cell surface receptors, quiescent endothelial cells are activated, resulting in cell proliferation and the secretion of proteases, such as MMPs, which contribute to angiogenesis by degrading basement membrane and other ECM components, allowing endothelial cells to detach and migrate into new tissue. The endothelial cell released proteases also have the effect of freeing ECM-bound proangiogenic, thus further augmenting angiogenesis.

The present invention provides compositions and methods that target the multiple aspects of the molecular and cellular events that underlie tumor associated angiogenesis. In particular, the present invention provides a modified anthrax lethal toxin that targets tumor associated angiogenesis by (1) direct cytotoxicity to cancer cells that have an activated MAP kinase pathway; (2) preventing the secretion of proangiogenic factors (e.g., IL-8) by tumor cells, regardless of activation of the MAP kinase pathway; and (3) direct cytotoxicity to activated endothelial cells. As detailed herein, the selectivity and effectiveness of the compositions of this invention in inhibiting tumor associated angiogenesis rests in part on the selective activation of these compositions by proteolysis of these compositions by tumor and activated endothelial proteases. Once proteolyzed, the compositions of the invention enter tumor and endothelial cells to effect inhibition of tumor associated angiogenesis.

### II. Definitions

The term “cancer” refers to human and animal cancers and carcinomas, sarcomas, adenocarcinomas, lymphomas, leukemias, solid and lymphoid cancers, etc. Examples of different types of cancer include, but are not limited to, prostate cancer, renal cancer (i.e., renal cell carcinoma), bladder cancer, lung cancer, breast cancer, thyroid cancer, liver cancer (i.e., hepatocarcinoma), pleural cancer, pancreatic cancer, ovarian cancer, uterine cancer, cervical cancer, testicular cancer, colon cancer, anal cancer, pancreatic cancer, bile duct cancer, gastrointestinal carcinoid tumors, esophageal cancer, gall bladder cancer, rectal cancer, appendix cancer, small intestine cancer, stomach (gastric) cancer, cancer of the central nervous system, skin cancer, choriocarcinoma; head and neck cancer, blood cancer, osteogenic sarcoma, fibrosarcoma, neuroblastoma, glioma, melanoma, B-cell lymphoma, non-Hodgkin's lymphoma, Burkitt's lymphoma, Small Cell lymphoma, Large Cell lymphoma, monocytic leukemia, myelogenous leukemia, acute lymphocytic leukemia, acute myelocytic leukemia, and multiple myeloma.

The term “endothelial” cell or “endothelium” refers generally to the thin layer of cells that line the interior surface of body cavities, blood vessels, and lymph vessels, thus forming an interface between, e.g., circulating blood in the lumen and the rest of a vessel wall. Examples of markers that are expressed on endothelial cells include, but are not limited to, 7B4 antigen, ACE (angiotensin-converting enzyme), BNH9/BNF13, CD31 (PECAM-1), CD34, CD54 (ICAM-1), CD62P (p-Selectin GMP140), CD105 (Endoglin), CD146 (P1H12), D2-40, E-selectin, EN4, Endocan, Endoglyx-1, Endomucin, Endosialin (tumor endothelial marker 1, TEM-1, FB5), Eotaxin-3, EPAS1 (Endothelial PAS domain protein 1), Factor VIII related antigen, FB21, Flk-1 (VEGFR-2), Flt-1 (VEGFR-1), GBP-1 (guanylate-binding protein-1), GRO-alpha, Hex, ICAM-2 (intercellular adhesion molecule 2), LYVE-1, MECA-32, MECA-79, Nucleolin, PAL-E, sVCAM-1, TEM1 (Tumor endothelial marker 1), TEM5 (Tumor endothelial marker 5), TEM7 (Tumor endothelial marker 7), TEM8 (Tumor endothelial marker 8), Thrombomodulin (TM, CD141), VCAM-1 (vascular cell adhesion molecule-1) (CD106), VE-cadherin (CD144), VEGF (Vascular endothelial growth factor), and vWF (von Willebrand factor).

The term “tumor associated angiogenesis” refers generally to the formation of vasculature to provide a blood supply to a tumor. As explained in greater detail herein, it is known that tumor associated angiogenesis entails complex interactions between a tumor and many different cells types, including but not limited to, endothelial cells, pericytes, and smooth muscle cells.

The term “tumor associated endothelial cell” refers generally to endothelial cells that form part of the vasculature which supplies blood to a tumor. Frequently, this vasculature arises as a result of tumor associated angiogenesis as described herein.

The terms “overexpress,” “overexpression,” or “overexpressed” interchangeably refer to a gene that is transcribed or translated at a detectably greater level, frequently in the context of a cancer cell or a stimulated endothelial cell, in comparison to a normal cell or non-stimulated or quiescent endothelial cell. In the present invention, overexpression can therefore refer to both overexpression of MMP or plasminogen activator or plasminogen activator receptor protein and RNA, as well as local overexpression due to altered protein trafficking patterns and/or augmented functional activity. Overexpression can result, e.g., from selective pressure in culture media, transformation, activation of endogenous genes, or by addition of exogenous genes. Overexpression can be detected using conventional techniques for detecting protein (e.g., ELISA, Western blotting, immunofluorescence, immunohistochemistry, immunoassays, cytotoxicity assays, growth inhibition assays, enzyme assays, gelatin zymography, etc.) or mRNA (e.g., RT-PCR, PCR, hybridization, etc.). One skilled in the art will know of other techniques suitable for detecting overexpression of MMP or plasminogen activator or plasminogen activator receptor protein or mRNA. For example, cancerous cells or stimulated endothelial cells can overexpress such proteins or RNAs at a level of at least about 5%, 10%, 15%, 20%, 25%, 30%, 35%, 40%, 45%, 50%, 55%, 60%, 65%, 70%, 75%, 80%, 85%, 90%, or 95% in comparison to corresponding normal, non-cancerous cells, or non-stimulated or quiescent endothelial cells. Cancerous cells or stimulated endothelial cells can also have at least about a 1-fold, 2-fold, 3-fold, 4-fold, 5-fold, 6-fold, or 7-fold higher level of MMP or plasminogen activator system protein transcription or translation in comparison to normal, non-cancerous cells, or non-stimulated or quiescent endothelial cells. In some cells, the expression of these proteins is very low or undetectable. As such, expression includes no expression, i.e., expression that is undetectable or insignificant.

Examples of cells overexpressing a MMP include the tumor cell lines, fibrosarcoma HT1080, melanoma A2058, and breast cancer MDA-MB-23 1. An example of a cell which does not overexpress a MMP is the non-tumor cell line Vero. An example of a cell that overexpresses a plasminogen activator receptor are the uPAR overexpressing cell types HeLa, A2058, and Bowes. An example of a cell which does not overexpress a plasminogen activator receptor is the non-tumor cell line Vero. An example of a cells that overexpress a tissue type plasminogen activator are cell types human melanoma Bowes and human primary vascular endothelial cells.

It will be appreciated by the skilled artisan that while cells overexpressing MMPs or plasminogen activator system proteins, such as cancer cells, will be targeted by the PA and LF compositions of the invention, some non-diseased cells which normally do not express these proteases are stimulated under various physiological conditions to express MMPs or plasminogen activator system proteins, and thus are targeted. Moreover, cells which otherwise express basal levels of these proteins will also be targeted.

“Apoptosis” refers generally to a process of programmed cell death and involves a series of ordered molecular events leading to characteristic changes in cell morphology and death, as distinguished from general cell death or necrosis that results from exposure of cells to non-specific toxic events such as metabolic poisons or ischemia. Cells undergoing apoptosis show characteristic morphological changes such as chromatin condensation and fragmentation and breakdown of the nuclear envelope. As apoptosis proceeds, the plasma membrane is seen to form blebbings, and the apoptotic cells are either phagocytosed or else break up into smaller vesicles which are then phagocytosed. Typical assays used to detect and measure apoptosis include microscopic examination of cellular morphology, TUNEL assays for DNA fragmentation, caspase activity assays, annexin-V externalization assays, and DNA laddering assays, among others. Apoptotic cells can be quantified by FACS analysis of cells stained with propidium iodide for DNA hypoploidy. It is well known to the skilled artisan that the process of apoptosis is controlled by a diversity of cell signals which includes extracellular signals such as hormones, growth factors, cytokines, and nitric oxide, among others. These signals may positively or negatively induce apoptosis. Other effectors of apoptosis include oncogenes (e.g., c-myc) and exposure of cancer cells to chemotherapeutic agents, among other examples.

“Inducing apoptosis” or “inducer of apoptosis” refers to an agent or process which causes a cell to undergo the program of cell death described above for apoptosis.

As used herein, the term “administering” means oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc.

By “therapeutically effective amount or dose” or “therapeutically sufficient amount or dose” herein is meant a dose that produces therapeutic effects for which it is administered.

The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins) and as further described herein.

### III. Anthrax Toxin

The symptoms of many bacterial diseases are due largely to the actions of toxic proteins released by the bacteria. Diphtheria toxin (DT) and Pseudomonas exotoxin A (PE) are two such well-known toxins secreted by the pathogenic bacterium Corynebacterium diphtheriae and the opportunistic pathogen Pseudomonas aeruginosa (Liu, S. and Leppla, S. H., Mol. Cell, 12:603-613 (2003)). After binding and entering mammalian cells, DT and PE catalyze the adenosine diphosphate (ADP)-ribosylation and inactivation of elongation factor 2 (EF2), leading to protein synthesis inhibition and cell death (Collier, R. J., Toxicon, 39:1793-1803 (2001); Liu, S., et al., Mol. Cell. Biol., 24:9487-9497 (2004)). The powerful lethal action of these toxins has been exploited extensively in the past two decades to target cancer cells by fusing the toxins with antibodies or growth factors that can selectively recognize antigens or receptors on cancer cells. These efforts have resulted in the first FDA-approved “immunotoxin”, DAB₃₈₉IL2 (denileukin diftitox or Ontak), a fusion of DT catalytic and translocation domains and IL2 (interleukin 2), for treatment of persistent or recurrent T-cell lymphoma (Olsen, E., et al., J. Clin. Oncol., 19:376-388 (2001)). With the rapid progress in understanding the structures and functions of anthrax lethal toxin (LT), an important virulence factor secreted by Bacillus anthracis, LT has been identified as a bacterial toxin having a completely different mode of action that can be used for tumor targeting (Liu, S, and Leppla, S. H., Mol. Cell, 12:603-613 (2003)).

Anthrax toxin is a three-part toxin secreted by Bacillus anthracis consisting of protective antigen (PA, 83 kDa), lethal factor (LF, 90 kDa) and edema factor (EF, 89 kDa), which are individually non-toxic (see Leppla, S. H. (1991) The anthrax toxin complex, p. 277-302. In J. E. Alouf and J. H. Freer (ed.), Sourcebook of bacterial protein toxins. Academic Press, London, UK; Leppla, S. H. Anthrax toxins, Handb. Nat. Toxins 8:543-572 (1995). To manifest cytotoxicity to mammalian cells, PA binds to the cell surface receptors tumor endothelium marker 8 (TEM8) and capillary morphogenesis gene 2 product (CMG2). PA is proteolytically activated by cell surface furin protease by cleavage at the sequence RKKR₁₆₇, leaving the carboxyl-terminal 63 kDa fragment (PA63) bound to the cell surface, resulting in the formation of the active PA63 heptamer and PA20, a 20 kDa N-terminal fragment, which is released into the medium. The PA63 heptamer then binds and translocates LF into the cytosol of the cell to exert its cytotoxic effects (Leppla, S. H., The Comprehensive Sourcebook of Bacterial Protein Toxins, 323-347 (2006)). An NCI60 anticancer drug screen (Shoemaker, 2006) identified LF cellular targets as the mitogen-activated protein kinase kinases (MEK) 1 and 2 (Duesbery, N. S., et al., Science, 280:734-737 (1998)). Later, the LF targets were extended to include MEK1 through 7, with the exception of MEK5 (Vitale, G., et al., Biochem. Biophys. Res. Commun., 248:706-711 (1998); Vitale, G., et al., Biochem. J. 352 Pt 3:739-745 (2000)). LF is a metalloproteinase which enzymatically cleaves and inactivates these MEKs and thus efficiently blocks three key mitogen-activated protein kinase (MAPK) pathways, including the ERK, p38, and Jun N-terminus kinase (JNK) pathways (Baldari, C. T., et al., Trends Immunol. 27:434-440 (2006)).

The PA63 heptamer is also able to bind EF. The combination of PA+EF, named edema toxin, disables phagocytes and probably other cells, due to the intracellular adenylate cyclase activity of EF (see, Klimpel, et al., Mol. Microbiol. 13:1094-1100 (1994); Leppla, S. H., et al., Bacterial Protein Toxins, p. 111-112 (1988) Gustav Fischer, New York, N.Y; Leppla, S. H., Proc. Natl. Acad. Sci. USA., 79:3162-3166 (1982)).

LF and EF have substantial sequence homology in amino acid (aa) 1-250, and a mutagenesis study showed this region constitutes the PA-binding domain (Leppla (1995) Anthrax toxins, Handb. Nat. Toxins 8:543-572; Quinn et al., J. Biol. Chem., 166:20124-20130 (1991)). Systematic deletion of LF fusion proteins containing the catalytic domain of Pseudomonas exotoxin A established that LF aa 1-254 (LFn) are sufficient to achieve translocation of “passenger” polypeptides to the cytosol of cells in a PA-dependent process (see Arora et al., J. Biol. Chem. 267:15542-15548 (1992); Arora et al., J. Biol. Chem. 268:3334-3341 (1993)). Accordingly, the term “LFn”, as used herein, refers to a fragment of LF that retains the ability to bind PA and comprising amino acids 1-254. A highly cytotoxic LFn fusion to the ADP-ribosylation domain of Pseudomonas exotoxin A, named FP59, has been developed (Arora et al., J. Biol. Chem. 268: 3334-3341 (1993)). When combined with PA, FP59 kills any cell type which contains receptors for PA by the mechanism of inhibition of initial protein synthesis through ADP ribosylating inactivation of elongation factor 2 (EF-2), whereas native LF is highly specific for macrophages (Leppla, Anthrax toxins, Handb. Nat. Toxins 8:543-572 (1995)). For this reason, FP59 is an example of a potent therapeutic agent when specifically delivered to the target cells with a target-specific PA.

The crystal structure of PA at 2.1 Å was solved by X-ray diffraction (PDB accession 1ACC) (Petosa et al., Nature 385:833-838 (1997)). PA is a tall, flat molecule having four distinct domains that can be associated with functions previously defined by biochemical analysis. Domain 1 (aa 1-258) contains two tightly bound calcium ions, and a large flexible loop (aa 162-175) that includes the sequence RKKR₁₆₇, which is cleaved by furin during proteolytic activation. Domain 2 (aa 259-487) contains several very long B-strands and forms the core of the membrane-inserted channel. It is also has a large flexible loop (aa 303-319) implicated in membrane insertion. Domain 3 (aa 488-595) has no known function. Domain 4 (aa 596-735) is loosely associated with the other domains and is involved in receptor binding. Because cleavage at RKKR₁₆₇ is absolutely required for the subsequent steps in toxin action, it was of great interest to engineer it to the cleavage sequences of some disease-associated proteases, such as matrix metalloproteinases (MMPs) and plasminogen activators (e.g., t-PA, u-PA, and uPAR; see, e.g., Romer et al., APMIS 107:120-127 (1999)), which are typically overexpressed in tumors.

A anticancer drug screen (NCI60) also revealed that LT is selectively toxic to many human melanoma cell lines, indicating that LT may be a useful therapeutic agent for human melanomas (Koo, H. M., et al., Proc. Natl. Acad. Sci., 99:3052-3057 (2002)). This selective cytotoxicity of LT to human melanomas was later linked to a BRAF-activating mutation occurring in the melanomas, an important discovery made by the Sanger Institute's Cancer Genome Project (Davies, H., et al., Nature, 417:949-954 (2002)). In this study, Davies and colleagues demonstrated that about 70% of human melanomas and a smaller fraction of other human cancer types contain a BRAF valine⁶⁰⁰ to glutamic acid mutation (V600E). BRAF is a serine/threonine kinase immediately upstream of MEK1/2 in the cascade of the ERK MAPK pathway. This mutation involves replacement of a neutral amino acid with a negatively charged one that mimics the phosphorylation of threonine⁵⁹⁹ and serine⁶⁰² in the activating loop and thus locks the molecule in the ‘on’ position (Wan, P. T., et al., Cell, 116:855-867 (2004)). Human melanomas with the oncogenic BRAF V600E mutation are dependent on the constitutive activation of the ERK pathway for survival. Thus, it was shown that human melanomas with the BRAF mutation were sensitive to LT, while those without the mutation were generally resistant (Abi-Habib, R. J., et al., Mol. Cancer. Ther., 4:1303-1310 (2005)). The anti-melanoma efficacy of LT was further recapitulated in vivo (Abi-Habib, R. J., et al., Clin. Cancer Res., 12:7437-7443 (2006)). However, LT, a major virulence factor of B. anthracis, has recognized in vivo toxicity, and thus might not be safe to use in human cancer patients (Moayeri, M., et al., J. Clin. Invest., 112:670-682 (2003)). Therefore, the development of an attenuated and tumor specific version of LT would be beneficial.

The unique requirement for PA proteolytic activation on the target cell surface provides a way to re-engineer this protein to make its cleavage dependent on proteases that are enriched in tumor tissues. To this end, we previously generated PA mutants requiring activation by matrix metalloproteinascs (MMPs) (Liu, S., et al., Cancer Res., 60:6061-6067 (2000)). MMPs are overproduced by tumor tissues and implicated in cancer cell growth, angiogenesis, and metastasis (Egeblad, M. and Werb, Z., Nat. Rev. Cancer, 2:161-174 (2002)). However, unlike furin, which is ubiquitously expressed, MMPs are restricted to only a small number of normal cells. Thus, we hypothesized that MMP-activated LT should have higher specificity to tumors. We show herein that the MMP-activated LT not only exhibits much lower toxicity than wild-type LT to mice, but also shows higher toxicity to human tumors in the tumor xenograft models. This is attributed, in part, to the unexpected greater bioavailability of MMP-activated PA protein in circulation. Moreover, we unexpectedly found that the MMP-activated LT has potent anti-tumor activity not only to human melanomas with the BRAF V600E mutation, but also to a wide range of other tumor types, regardless of the BRAF mutation status. This potent generic anti-tumor activity is due to the targeting of tumor vasculature and angiogenic processes.

### IV. MMPs and Plasminogen Activators

MMPs and plasminogen activators are families of enzymes that play a leading role in both the normal turnover and pathological destruction of the extracellular matrix, including tissue remodeling (Birkedal-Hansen, H., Curr. Opin. Cell Biol., 7:728-735 (1995); Alexander, C. M., et al., Development, 122:1723-1736 (1996)), angiogenesis (Schnaper, H. W., et al., J. Cell Physiol., 156:235-246 (1993)), tumor invasion and metastasis formation. The members of the MMP family are multidomain, zinc-containing, neutral endopeptidases and include the collagenases, stromelysins, gelatinases, and membrane-type metalloproteinases (Birkedal-Hansen, H., Curr. Opin. Cell Biol, 7:728-735 (1995)). It has been well documented in recent years that MMPs and proteins of the plasminogen activation system, e.g., plasminogen activator receptors and plasminogen activators, are overexpressed in a variety of tumor tissues and tumor cell lines and are highly correlated to the tumor invasion and metastasis (Crawford, H. C., et al., Invasion Metastasis, 14:234-245 (1995); Garbisa, S., et al., Cancer Res., 47:1523-1528 (1987); Himelstein, B. P., et al., Invest. Methods, 14:246-258 (1995); Juarez, J., et al., Int. J. Cancer, 55:10-18 (1993); Kohn, E. C., et al., Cancer Res., 55:1856-1862 (1995); Levy, A. T., et al., Cancer Res., 51:439-444 (1991); Mignatti, P., et al., Physiol. Rev., 73:161-195 (1993); Montgomery, A. M., et al., Cancer Res., 53:693-700 (1993); Stetler-Stevenson, W. G., et al., Annu. Rev. Cell Biol., 9:541-573 (1993); Stetler-Stevenson, W. G., Invest. Methods, 14:4664-4671 (1995); Davidson, B., et al., Gynecol. Oncol., 73:372-382 (1999); Webber, M. M., et al., Carcinogenesis, 20:1185-1192 (1999); Johansson, N., et al., Am. J. Pathol., 154:469-480 (1999); Ries, C., et al., Clin. Cancer Res., 5:1115-1124 (1999); Zeng, Z. S., et al., Carcinogenesis, 20:749-755 (1999); Gokaslan, Z. L., et al., Clin. Exp. Metastasis, 16:721-728 (1998); Forsyth, P. A., et al., Br. J. Cancer, 79:1828-1835 (1999); Ozdemir, E., et al., J. Urol., 161:1359-1363 (1999); Nomura, H., et al., Cancer Res., 55:3263-3266 (1995); Okada, Y., et al., Proc. Natl. Acad. Sci. USA, 92:2730-2734 (1995); Sato, H., et al., Nature, 370:61-65 (1994); Chen, W. T., et al., Ann. NY Acad. Sci., 878:361-371 (1999); Sato, T., et al., Br. J. Cancer, 80:1137-43 (1999); Polette, M., et al., Int. J. Biochem. Cell Biol., 30:1195-1202 (1998); Kitagawa, Y., et al., J. Urol., 160:1540-1545; Nakada, M., et al., Am. J. Pathol., 154:417-428 (1999); Sato, H., et al., Thromb. Haemost, 78:497-500 (1997)).

Among the MMPs, MMP-2 (gelatinase A), MMP-9 (gelatinase B) and membrane-type 1 MMP (MT1-MMP) are reported to be most related to invasion and metastasis in various human cancers (Crawford, H. C., et al., Invasion Metastasis, 14:234-245 (1995); Garbisa, S., et al., Cancer Res., 47:1523-1528 (1987); Himelstein, B. P., et al., Invest. Methods, 14:246-258 (1995); Juarez, J., et al., Int. J. Cancer, 55:10-18 (1993); Kohn, E. C., et al., Cancer Res., 55:1856-1862 (1995); Levy, A. T., et al., Cancer Res., 51:439-444 (1991); Mignatti, P., et al., Physiol. Rev., 73:161-195 (1993); Montgomery, A. M., et al., Cancer Res., 53:693-700 (1993); Stetler-Stevenson, W. G., et al., Annu. Rev. Cell Biol., 9541-9573 (1993); Stetler-Stevenson, W. G., Invest. Methods, 14:4664-4671 (1995); Davidson, B., et al., Gynecol. Oncol., 73:372-382 (1999); Webber, M. M., et al., Carcinogenesis, 20:1185-1192 (1999); Johansson, N., et al., Am. J. Pathol., 154:469-480 (1999); Ries, C., et al., Clin. Cancer Res., 5:1115-1124 (1999); Zeng, Z. S., et al., Carcinogenesis, 20:749-755 (1999); Gokaslan, Z. L., et al., Clin. Exp. Metastasis, 16:721-728 (1998); Forsyth, P. A., et al., Br. J. Cancer, 79:1828-1835 (1999); Ozdemir, E., et al., J. Urol., 161:1359-1363 (1999); Nomura, H., et al., Cancer. Res., 55:3263-3266 (1995); Okada, Y., et al., Proc. Natl. Acad. Sci. USA, 92:2730-2734 (1995); Sato, H., et al., Nature, 370:61-65 (1994); Chen, W. T., et al., Ann. NY Acad. Sci., 878:361-371 (1999); Sato, T., et al., Br J Cancer, 80:1137-43 (1999); Polette, M., et al., Int. J. Biochem. Cell Biol., 30:1195-1202 (1998); Kitagawa, Y., et al., J. Urol., 160:1540-1545; Nakada, M., et al., Am. J. Pathol., 154:417-428 (1999); Sato, H., et al., Thromb. Haemost, 78:497-500 (1997)). The important role of MMPs during tumor invasion and metastasis is to break down tissue extracellular matrix and dissolution of epithelial and endothelial basement membranes, enabling tumor cells to invade through stroma and blood vessel walls at primary and secondary sites. MMPs also participate in tumor neoangiogenesis and are selectively upregulated in proliferating endothelial cells in tumor tissues (Schnaper, H. W., et al., J. Cell Physiol., 156:235-246 (1993); Chambers, A. F., et al., J. Natl. Cancer Inst., 89:1260-1270 (1997)). Furthermore, these proteases can contribute to the sustained growth of established tumor foci by the ectodomain cleavage of membrane-bound pro-forms of growth factors, releasing peptides that are mitogens for tumor cells and/or tumor vascular endothelial cells (Arribas, J., et al., J. Biol. Chem., 271:11376-11382 (1996); Suzuki, M., et al., J. Biol. Chem., 272:31730-31737 (1997)).

However, catalytic manifestations of MMP and plasminogen activators are highly regulated. For example, the MMPs are expressed as inactive zymogen forms and require activation before they can exert their proteolytic activities. The activation of MMP zymogens involves sequential proteolysis of N-terminal propeptide blocking the active site cleft, mediated by proteolytic mechanisms, often leading to an autoproteolytic event (Springman, E. B., et al., Proc. Natl. Acad. Sci. USA, 873364-368 (1990); Murphy, G., et al., APMIS, 107:38-44 (1999)). Second, a family of proteins, the tissue inhibitors of metalloproteinases (TIMPs), are correspondingly widespread in tissue distribution and function as highly effective MMP inhibitors (Ki˜10⁻¹⁰ M) (Birkedal-Hansen, H., et al., Crit. Rev. Oral Biol. Med., 4:197-250 (1993)). Though the activities of MMPs are tightly controlled, invading tumor cells that utilize the MMPs degradative capacity somehow circumvent these negative regulatory controls, but the mechanisms are not well understood.

The contributions of MMPs in tumor development and metastatic process lead to the development of novel therapies using synthetic inhibitors of MMPs (Brown, P.D., Adv. Enzyme Regul., 35:293-301 (1995); Wojtowicz-Praga, S., et al., J. Clin. Oncol., 16:2150-2156 (1998); Drummond, A. H., et al., Ann. NY Acad. Sci., 30:228-235 (1999)). Among a multitude of synthetic inhibitors generated, Marimastat is already clinically employed in cancer treatment (Drummond, A. H., et al., Ann. NY Acad. Sci., 30:228-235 (1999)).

As an alternate to the use of MMP inhibitors, we used a novel strategy using modified PAs which could only be activated by MMPs or plasminogen activators to specially kill MMP- or and plasminogen activator-expressing tumor cells. PA mutants are constructed in which the furin recognition site is replaced by sequences susceptible to cleavage by MMPs or and plasminogen activators. When combined with LF or an LF fusion protein comprising the PA binding site, these PA mutants are specifically cleaved by cancer cells, exposing the LF binding site and translocating the LF or LF fusion protein into the cell, thereby specifically delivering compounds, e.g., a therapeutic or diagnostic agent, to the cell (see WO 01/21656).

Proteolytic degradation of the extracellular matrix plays a crucial role both in cancer invasion and non-neoplastic tissue remodeling, and in both cases it is accomplished by a number of proteases. Best known are the plasminogen activation system that leads to the formation of the serine protease plasmin, and a number of matrix metalloproteinase, including collagenases, gelatinases and stromelysins (Dano, K., et al., APMIS, 107:120-127 (1999)). The close association between MMP and plasminogen activator overexpression and tumor metastasis has been noticed for two decades. For example, the contributions of MMPs in tumor development and metastatic processes lead to the development of novel therapies using synthetic inhibitors of MMPs (Brown, P.D., Adv. Enzyme Regul., 35:293-301 (1995); Wojtowicz-Praga, S., et al., J. Clin. Oncol., 16:2150-2156 (1998); Drummond, A. H., et al., Ann. NY Acad. Sci., 30:228-235 (1999)). However, these inhibitors only slow growth and do not eradicate the tumors. Mutant PA molecules in which the furin cleavage site is replaced by an MMP or plasminogen activator target site can be used to deliver compounds such as toxins to the cell, thereby killing the cell. The compounds have the ability to bind PA through their interaction with LF and are translocated by PA into the cell. The PA and LF-comprising compounds are administered to cells or subjects, preferably mammals, more preferably humans, using techniques known to those of skill in the art. Optionally, the PA and LF-comprising compounds are administered with a pharmaceutically acceptable carrier.

The compounds typically are either native LF or an LF fusion protein, i.e., those that have a PA binding site (approximately the first 250 amino acids of LF, Arora et al., J. Biol. Chem. 268:3334-3341 (1993)) fused to another polypeptide or compound so that the protein or fusion protein binds to PA and is translocated into the cell, causing cell death (e.g., recombinant toxin FP59, anthrax toxin lethal factor residue 1-254 fusion to the ADP-ribosylation domain of Pseudomonas exotoxin A). The fusion is typically chemical or recombinant. The compounds fused to LF include, e.g., therapeutic or diagnostic agent, e.g., native LF, a toxin, a bacterial toxin, shiga toxin, A chain of diphtheria toxin, Pseudomonas exotoxin A, a protease, a growth factor, an enzyme, a detectable moiety, a chemical compound, a nucleic acid, or a fusion polypeptide, etc.

The mutant PA molecules of the invention can be further targeted to a specific cell by making mutant PA fusion proteins. In these mutant fusion proteins, the PA receptor binding domain is replaced by a protein such as a growth factor or other cell receptor ligand specifically expressed on the cells of interest. In addition, the PA receptor binding domain may be replaced by an antibody that binds to an antigen specifically expressed on the cells of interest.

These proteins provide a way to specifically kill tumor cells without serious damage to normal cells. This method can also be applied to non-cancer inflammatory cells that contain high amounts of cell-surface associated MMPs or plasminogen activators. These PA mutants are thus useful as therapeutic agents to specifically kill tumor cells.

We constructed two PA mutants, PA-L1 and PA-L2, in which the furin recognition site is replaced by sequences susceptible to cleavage by MMPs, especially by MMP-2 and MMP-9. When combined with FP59, these two PA mutant proteins specifically killed MMP-expressing tumor cells, such as human fibrosarcoma HT1080 and human melanoma A2058, but did not kill MMP non-expressing cells. Cytotoxicity assay in the co-culture model, in which all the cells were in the same culture environment and were equally accessible to the toxins in the supernatant, showed PA-L1 and PA-L2 specifically killed only MMP-expressing tumor cells HT1080 and A2058, not Vero cells. This result demonstrated activation processing of PA-L1 and PA-L2 mainly occurred on the cell surfaces and mostly contributed by the membrane-associated MMPs, so the cytotoxicity is restricted to MMP-expressing tumor cells. TIMPs are widely present in extracellular milieu and inhibit MMP activity in supernatants. PA proteins bind to the cells very quickly with maximum binding happened within 60 min. In contrast to secreted MMPs, membrane-associated MMPs express their proteolytic activities more efficiently by anchoring on cell membrane and enjoying two distinct advantageous properties, which are highly focused on extracellular matrix substrates and more resistant to proteinase inhibitors present in extracellular milieu.

Recently it has been shown that physiological concentrations of plasmin can activate both MMP-2 and MMP-9 on cell surface of HT1080 by a mechanism independent of MMP or acid proteinase activities (Mazzieri, R., et al., EMBO J., 16:2319-2332 (1997)). In contrast, in soluble phase, plasmin degrades both MMP-2 and MMP-9 (Mazzieri, R., et al., EMBO J., 16:2319-2332 (1997)). Thus, plasmin may provide a mechanism keeping gelatinase activities on cell surface to promote cell invasion. It has been well established MT1-MMP functions as both activator and receptor of MMP-2, but has no effect on MMP-9 (see Polette, M., et al., Int. J. Biochem. Cell Biol., 30:1195-1202 (1998); Sato, H., et al., Thromb. Haemost, 78:497-500 (1997) for review). A MMP-2/TIMP-2 complex binds to MT1-MMP on cell surface, which serves as a high affinity site, then be proteolytically activated by an adjacent MT1-MMP, which serves as an activator. For MMP activities involved in tumor invasion and metastasis are localized and/or modulated on the cell surface in insoluble phase, this makes MMPs an ideal target for tumor tissues.

It was originally thought that the role of MMPs and plasminogen activators was simply to break down tissue barriers to promote tumor invasion and metastasis. As we show here, MMPs also participate in tumor neoangiogenesis and are selectively upregulated in proliferating endothelial cells. Therefore, these modified bacterial toxins have advantageous properties that target not only tumor cells themselves but also the dividing vascular endothelial cells which are essential to neoangiogenesis in tumor tissues. Therefore, the MMP targeted toxins may also kill tumor cells by starving the cells of necessary nutrients and oxygen.

The mutant PA molecules of the invention can also be specifically targeted to cells using mutant PA fusion proteins. In these fusion proteins, the receptor binding domain of PA is replaced with a heterologous ligand or molecule such as an antibody that recognizes a specific cell surface protein. PA protein has four structurally distinct domains for performing the functions of receptor binding and translocation of the catalytic moieties across endosomal membranes (Petosa, C., et al., Nature, 385:833-838 (1997)). Domain 4 is the receptor-binding domain and has limited contacts with other domains (Petosa, C., et al., Nature, 385:833-838 (1997)). Therefore, PA can be specifically targeted to alternate receptors or antigens specifically expressed by tumors by replacing domain 4 with the targeting molecules, such as single-chain antibodies or a cytokines used by other immunotoxins (Thrush, G. R., et al., Annu. Rev. Immunol., 14:49-71 (1996)). For example, PA-L1 and PA-L2 are directed to alternate receptors, such as GM-CSF receptor, which is highly expressed in leukemias cells and solid tumors including renal, lung, breast and gastrointestinal carcinomas (Thrush, G. R., et al., Annu. Rev. Immunol., 14:49-71 (1996)). It should be highly expected that the combination of these two independent targeting mechanism should allow tumors to be more effectively targeted, and side effects such as hepatotoxicity and vascular leak syndrome should be significantly reduced.

With respect to the plasminogen activation system, two plasminogen activators are known, the urokinase-type plasminogen activator (uPA) and the tissue-type plasminogen activator (tPA) (Dano, K., et al., APMIS, 107:120-127 (1999)). uPA is a 52 kDa serine protease which is secreted as an inactive single chain proenzyme (pro-uPA) (Nielsen, L. S., et al., Biochemisty, 21:6410-6415 (1982); Petersen, L. C., et al., J. Biol. Chem., 263:11189-11195 (1988)). The binding domain of pro-uPA is the epidermal growth factor-like amino-terminal fragment (ATF; aa 1-135, 15 kDa) that binds with high affinity (Kd=0.5 mM) to urokinase-type plasminogen activator receptor (uPAR) (Cubellis, M. V., et al., Proc. Natl. Acad. Sci. U.S.A., 86:4828-4832 (1989)), a GPI-linked receptor. uPAR is a 60 kDa three domain glycoprotein whose N-terminal domain 1 contains the high affinity binding site for ATF of pro-uPA (Ploug, M., et al., J. Biol. Chem., 266:1926-1933 (1991); Behrendt, N., et al., J. Biol. Chem., 266:7842-7847 (1991)). uPAR is overexpressed on a variety of tumors, including monocytic and myelogenous leukemias (Lanza, F., et al., Br. J. Haematol., 103:110-123 (1998); Plesner, T., et al., Am. J. Clin. Pathol., 102:835-841 (1994)), and cancers of the breast (Carriero, M. V., et al., Clin. Cancer Res., 3:1299-1308 (1997)), bladder (Hudson, M. A., et al., J. Natl. Cancer Inst., 89:709-717 (1997)), thyroid (Ragno, P., et al., Cancer Res., 58:1315-1319 (1998)), liver (De Petro, G., et al., Cancer Res., 58:2234-2239 (1998)), pleura (Shetty, S., et al., Arch. Biochem. Biophys., 356:265-279 (1998)), lung (Morita, S., et al., Int. J. Cancer, 78:286-292 (1998)), pancreas (Taniguchi, T., et al., Cancer Res., 58:4461-4467 (1998)), and ovaries (Sier, C. F., et al., Cancer Res., 58:1843-1849 (1998)). Pro-uPA binds to uPAR by ATF, while the binding process does not block the catalytic, carboxyl-terminal domain. By association with uPAR, pro-uPA gets near to and subsequently activated by trace amounts of plasmin bound to the plasma membrane by cleavage of the single chain pro-uPA within an intra-molecular loop held closed by a disulfide bridge. Thus the active uPA consists of two chains (A+B) held together by this disulfide bond (Ellis, V., et al., J. Biol. Chem., 264:2185-2188 (1989)). Plasminogen is present at high concentration (1.5-2.0 μM) in plasma and interstitial fluids (Dano, K., et al., Adv. Cancer Res., 44:139-266 (1985)). Low affinity, high capacity binding of plasminogen to cell-surface proteins through the lysine binding sites of plasminogen kringles enhances considerably the rate of plasminogen activation by uPA (Ellis, V., et al., J. Biol. Chem., 264:2185-2188 (1989); Stephens, R. W., et al., J. Cell Biol., 108:1987-1995 (1989)). Active uPA has high specificity for the Arg560-Val561 bond in plasminogen, and cleavage between these residues gives rise to more plasmin that is referred to as “reciprocal zymogen activation” (Petersen, L. C., Eur. J. Biochem., 245:316-323 (1997)). The result of this system is efficient generation of active uPA and plasmin on cell surface. In this context, uPAR serves as a template for binding and localization of pro-uPA near to its substrate plasminogen on plasma membrane.

Unlike uPA, plasmin is a relatively non-specific protease, cleaving fibrin, as well as, many glycoproteins and proteoglycans of the extracellular matrix (Liotta, L. A., et al., Cancer Res., 41:4629-4636 (1981)). Therefore, cell surface bound plasmin mediates the non-specific matrix proteolysis which facilitates invasion and metastasis of tumor cells through restraining tissue structures. In addition, plasmin can activate some of the matrix metalloproteases which also degrade tissue matrix (Werb, Z., et al., N. Engl. J. Med., 296:1017-1023 (1977); DeClerck, Y. A., et al., Enzyme Protein, 49:72-84 (1996)). Plasmin can also activate growth factors, such as TGF-β, which may further modulate stromal interactions in the expression of enzymes and tumor neo-angiogenesis (Lyons, R. M., et al., J. Cell Biol., 106:1659-1665 (1988)). Plasminogen activation by uPA is regulated by two physiological inhibitors, plasminogen activator inhibitor-1 and 2 (PAI-1 and PAI-2) (Cubellis, M. V., et al., Proc. Natl. Acad. Sci. U.S.A., 86:4828-4832 (1989); Ellis, V., et al., J. Biol. Chem., 265:9904-9908 (1990); Baker, M. S., et al., Cancer Res., 50:4676-4684 (1990)), by formation 1:1 complex with uPA. Plasmin generated in the cell surface plasminogen activation system is relatively protected from its principle physiological inhibitor α2-antiplasmin (Ellis, V., et al., J. Biol. Chem., 266:12752-12758 (1991)).

Cancer invasion is essentially a tissue remodeling process in which normal tissue is substituted with cancer tissue. Accumulated data from preclinical and clinical studies strongly suggested that the plasminogen activation system plays a central role in the processes leading to tumor invasion and metastasis (Andreasen, P. A., et al., Int. J. Cancer, 72:1-22 (1997); Chapman, H. A., Curr. Opin. Cell Biol., 9:714-724 (1997); Schmitt, M., et al., Thromb. Haemost., 78:285-296 (1997)). High levels of uPA, uPAR, and PM-1 are associated with poor disease outcome (Schmitt, M., et al., Thromb. Haemost., 78:285-296 (1997)). In situ hybridization studies of tumor tissues has shown that usually cancer cells show highly expressed uPAR, while tumor stromal cells expressed pro-uPA, which subsequently binds to uPAR on the surface of cancer cells where it is activated and thereby generating plasmin (Pyke, C., et al., Am. J. Pathol., 138:1059-1067 (1991)). For the activation of pro-uPA is highly restricted to the tumor cell surface, it may be an ideal target for cancer treatment.

uPA and tPA possess an extremely high degree of structural similarity (Lamba, D., et al., J. Mol. Biol., 258:117-135 (1996); Spraggon, G., et al., Structure, 3:681-691 (1995)), share the same primary physiological substrate (plasminogen) and inhibitors (PAI-1 and PAI-2) (Collen, D., et al., Blood, 78:3114-3124 (1991)), and exhibit restricted substrate specificity. By using substrate phage display and substrate subtraction phage display approaches, recent investigations had identified substrates that discriminate between uPA and tPA, showing the consensus substrate sequences with high selectivity by uPA or tPA (Ke, S. H., et al., J. Biol. Chem., 272:20456-20462 (1997); Ke, S. H., et al., J. Biol. Chem., 272:16603-16609 (1997)). To exploit the unique characteristics of the uPA plasminogen system and anthrax toxin in the design of tumor cell selective cytotoxins, in the work described here, mutated anthrax PA proteins were constructed in which the furin site is replaced by sequences susceptible to specific cleavage by uPA. These uPAR/uPA-targeted PA proteins were activated selectively on the surface of uPAR-expressing tumor cells in the presence of pro-uPA, and caused internalization of a recombinant cytotoxin FP59 to selectively kill the tumor cells. Also, a mutated PA protein was generated which selectively killed tissue-type plasminogen activator expressing cells.

### V. Methods of Producing PA and LF Constructs

A. Construction of Nucleic Acids Encoding PA Mutants, LF, and PA and LF Fusion Proteins PA includes a cellular receptor binding domain, a translocation domain, and an LF binding domain. The PA polypeptides of the invention have at least a translocation domain and an LF binding domain. In the present invention, mature PA (83 kDa) is one preferred embodiment. In addition to full length recombinant PA, aminoterminal deletions up to the 63 kDa cleavage site or additions to the full length PA are useful. A recombinant form of processed PA is also biologically active and could be used in the present invention. PA fusion proteins in which the receptor binding domain has been deleted can also be constructed to target PA to specific cell types. Although the foregoing and the prior art describe specific deletion and structure-function analysis of PA, any biologically active form of PA can be used in the present invention.

Amino-terminal residues 1-254 of LF are sufficient for PA binding activity. Amino acid residues 199-253 may not all be required for PA binding activity. One embodiment of LF is amino acids 1-254 of native LF. Any embodiment that contains at least about amino acids 1-254 of native LF can be used in the present invention, for example, native LF. Nontoxic embodiments of LF are preferred.

PA and LF fusion proteins can be produced using recombinant nucleic acids that encode a single-chain fusion protein. The fusion protein can be expressed as a single chain using in vivo or in vitro biological systems. Using current methods of chemical synthesis, compounds can be also be chemically bound to PA or LF. The fusion protein can be tested empirically for receptor binding, PA or LF binding, and internalization using methods as set forth, for example in WO 01/21656 A2.

In addition, functional groups capable of forming covalent bonds with the amino- and carboxyl-terminal amino acids or side groups of amino acids are well known to those of skill in the art. For example, functional groups capable of binding the terminal amino group include anhydrides, carbodiimides, acid chlorides, and activated esters. Similarly, functional groups capable of forming covalent linkages with the terminal carboxyl include amines and alcohols. Such functional groups can be used to bind compound to LF at either the amino- or carboxyl-terminus. Compound can also be bound to LF through interactions of amino acid residue side groups, such as the SH group of cysteine (see, e.g., Thorpe et al., Monoclonal Antibody-Toxin Conjugates: Aiming the Magic Bullet, in Monoclonal Antibodies in Clinical Medicine, pp. 168-190 (1982); Waldmann, Science, 252:1657 (1991); U.S. Pat. Nos. 4,545,985 and 4,894,443). The procedure for attaching an agent to an antibody or other polypeptide targeting molecule will vary according to the chemical structure of the agent. As an example, a cysteine residue can be added at the end of LF. Since there are no other cysteines in LF, this single cysteine provides a convenient attachment point through which to chemically conjugate other proteins through disulfide bonds. Although certain of the methods of the invention have been described as using LF fusion proteins, it will be understood that other LF compositions having chemically attached compounds can be used in the methods of the invention.

Protective antigen proteins can be produced from nucleic acid constructs encoding mutants, in which the naturally occurring furin cleavage site has been replaced by an MMP or a plasminogen activator cleavage site. In addition, LF proteins, and LF and PA fusion proteins can also be expressed from nucleic acid constructs according to standard methodology. Those of skill in the art will recognize a wide variety of ways to introduce mutations into a nucleic acid encoding protective antigen or to construct a mutant protective antigen-encoding nucleic acid. Such methods are well known in the art (see Sambrook et al., Molecular Cloning, A Laboratory Manual (2nd ed. 1989); Kriegler, Gene Transfer and Expression: A Laboratory Manual (1990); and Current Protocols in Molecular Biology (Ausubel et al., eds., 1994)). In some embodiments, nucleic acids of the invention are generated using PCR. For example, using overlap PCR protective antigen encoding nucleic acids can be generated by substituting the nucleic acid subsequence that encodes the furin site with a nucleic acid subsequence that encodes a matrix metalloproteinase (MMP) site (e.g., GPLGMLSQ and GPLGLWAQ). Similarly, an overlap PCR method can be used to construct the protective antigen proteins in which the furin site is replaced by a plasminogen activator cleavage site (e.g., the uPA and tPA physiological substrate sequence PCPGRVVGG, the uPA favorite sequence PGSGRSA, the uPA favorite sequence PGSGKSA, or the tPA favorite sequence PQRGRSA) (see, e.g., WO 01/21656).

### B. Expression of LF, PA and LF and PA Fusion Proteins

To obtain high level expression of a nucleic acid (e.g., cDNA, genomic DNA, PCR product, etc. or combinations thereof) encoding a native (e.g., PA) or mutant protective antigen protein (e.g., PA-L1, PA-L2, PA-U1, PA-U2, PA-U3, PA-U4, etc.), LF, or a PA or LF fusion protein, one typically subclones the protective antigen encoding nucleic acid into an expression vector that contains a strong promoter to direct transcription, a transcription/translation terminator, and if for a nucleic acid encoding a protein, a ribosome binding site for translational initiation. Suitable bacterial promoters are well known in the art and described, e.g., in Sambrook et al. and Ausubel et al. Bacterial expression systems for expressing the protective antigen encoding nucleic acid are available in, e.g., E. coli, Bacillus sp., and Salmonella (Palva et al., Gene 22:229-235 (1983)). Kits for such expression systems are commercially available. Eukaryotic expression systems for mammalian cells, yeast, and insect cells are well known in the art and are also commercially available.

In some embodiments, protective antigen containing proteins are expressed in non-virulent strains of Bacillus using Bacillus expression plasmids containing nucleic acid sequences encoding the particular protective antigen protein (see, e.g., Singh, Y., et al., J. Biol. Chem., 264:19103-19107 (1989)). The protective antigen containing proteins can be isolated from the Bacillus culture using protein purification methods (see, e.g., Varughese, M., et al., Infect. Immun., 67:1860-1865 (1999)).

The promoter used to direct expression of a protective antigen encoding nucleic acid depends on the particular application. The promoter is preferably positioned about the same distance from the heterologous transcription start site as it is from the transcription start site in its natural setting. As is known in the art, however, some variation in this distance can be accommodated without loss of promoter function. The promoter typically can also include elements that are responsive to transactivation, e.g., Gal4 responsive elements, lac repressor responsive elements, and the like. The promoter can be constitutive or inducible, heterologous or homologous.

In addition to the promoter, the expression vector typically contains a transcription unit or expression cassette that contains all the additional elements required for the expression of the nucleic acid in host cells. A typical expression cassette thus contains a promoter operably linked, e.g., to the nucleic acid sequence encoding the protective antigen containing protein, and signals required for efficient expression and termination and processing of the transcript, ribosome binding sites, and translation termination. The nucleic acid sequence may typically be linked to a cleavable signal peptide sequence to promote secretion of the encoded protein by the transformed cell. Such signal peptides would include, among others, the signal peptides from bacterial proteins, or mammalian proteins such as tissue plasminogen activator, insulin, and neuron growth factor, and juvenile hormone esterase of Heliothis virescens. Additional elements of the cassette may include enhancers and, if genomic DNA is used as the structural gene, introns with functional splice donor and acceptor sites.

In addition to a promoter sequence, the expression cassette should also contain a transcription termination region downstream of the structural gene to provide for efficient termination and processing, if desired. The termination region may be obtained from the same gene as the promoter sequence or may be obtained from different genes.

The particular expression vector used to transport the genetic information into the cell is not particularly critical. Any of the conventional vectors used for expression in eukaryotic or prokaryotic cells may be used. Standard bacterial expression vectors include plasmids such as pBR322 based plasmids, pSKF, pET23D, and fusion expression systems such as GST and LacZ. Epitope tags can also be added to recombinant proteins to provide convenient methods of isolation, e.g., c-myc.

Expression vectors containing regulatory elements from eukaryotic viruses are typically used in eukaryotic expression vectors, e.g., SV40 vectors, papilloma virus vectors, and vectors derived from Epstein-Barr virus. Other exemplary eukaryotic vectors include pMSG, pAV009/A+, pMTO10/A+, pMAMneo-5, baculovirus pDSVE, and any other vector allowing expression of proteins under the direction of the SV40 early promoter, SV40 later promoter, metallothionein promoter, murine mammary tumor virus promoter, Rous sarcoma virus promoter, polyhedrin promoter, or other promoters shown to be effective for expression in eukaryotic cells.

Some expression systems have markers that provide gene amplification such as thymidine kinase, hygromycin B phosphotransferase, and dihydrofolate reductase. Alternatively, high yield expression systems not involving gene amplification are also suitable, such as using a baculovirus vector in insect cells, with a protective antigen encoding nucleic acid under the direction of the polyhedrin promoter or other strong baculovirus promoters.

The elements that are typically included in expression vectors also include a replicon that functions in E. coli, a gene encoding antibiotic resistance to permit selection of bacteria that harbor recombinant plasmids, and unique restriction sites in nonessential regions of the plasmid to allow insertion of heterologous sequences. The particular antibiotic resistance gene chosen is not critical, any of the many resistance genes known in the art are suitable. The prokaryotic sequences are preferably chosen such that they do not interfere with the replication of the DNA in eukaryotic cells, if necessary.

Standard transfection methods are used to produce bacterial, mammalian, yeast or insect cell lines that express large quantities of protein, which are then purified using standard techniques (see, e.g., Colley et al., J. Biol. Chem. 264:17619-17622 (1989); Guide to Protein Purification, in Methods in Enzymology, vol. 182 (Deutscher, ed., 1990)). Transformation of eukaryotic and prokaryotic cells are performed according to standard techniques (see, e.g., Morrison, J. Bact. 132:349-351 (1977); Clark-Curtiss & Curtiss, Methods in Enzymology 101:347-362 (Wu et al., eds. 1983).

Any of the well known procedures for introducing foreign nucleotide sequences into host cells may be used. These include the use of calcium phosphate transfection, polybrene, protoplast fusion, electroporation, liposomes, microinjection, plasma vectors, viral vectors and any of the other well known methods for introducing cloned genomic DNA, cDNA, synthetic DNA or other foreign genetic material into a host cell (see, e.g., Sambrook et al., supra). It is only necessary that the particular genetic engineering procedure used be capable of successfully introducing at least one gene into the host cell capable of expressing the protein of choice.

After the expression vector is introduced into the cells, the transfected cells are cultured under conditions favoring expression of the protective antigen containing protein, which is recovered from the culture using standard techniques identified below.

### VI. Purification of Polypeptides of the Invention

Recombinant proteins of the invention can be purified from any suitable expression system, e.g., by expressing the proteins in B. anthracis and then purifying the recombinant protein via conventional purification techniques (e.g., ammonium sulfate precipitation, ion exchange chromatography, gel filtration, etc.) and/or affinity purification, e.g., by using antibodies that recognize a specific epitope on the protein or on part of the fusion protein, or by using glutathione affinity gel, which binds to GST (see, e.g., Scopes, Protein Purification: Principles and Practice (1982); U.S. Pat. No. 4,673,641; Ausubel et al., supra; and Sambrook et al., supra). In some embodiments, the recombinant protein is a fusion protein with GST or Gal4 at the N-terminus. Those of skill in the art will recognize a wide variety of peptides and proteins that can be fused to the protective antigen containing protein to facilitate purification (e.g., maltose binding protein, a polyhistidine peptide, etc.).

A. Purification of Proteins from Recombinant Bacteria

Recombinant and native proteins can be expressed by transformed bacteria in large amounts, typically after promoter induction; but expression can be constitutive. Promoter induction with IPTG is one example of an inducible promoter system. Bacteria are grown according to standard procedures in the art. Fresh or frozen bacteria cells are used for isolation of protein.

Proteins expressed in bacteria may form insoluble aggregates (“inclusion bodies”). Several protocols are suitable for purification of inclusion bodies. For example, purification of inclusion bodies typically involves the extraction, separation and/or purification of inclusion bodies by disruption of bacterial cells, e.g., by incubation in a buffer of 50 mM Tris/HCl pH 7.5, 50 mM NaCl, 5 mM MgCl₂, 1 mM DTT, 0.1 mM ATP, and 1 mM PMSF. The cell suspension can be lysed using 2-3 passages through a French press, homogenized using a Polytron (Brinkman Instruments) or sonicated on ice. Alternate methods of lysing bacteria are apparent to those of skill in the art (see, e.g., Sambrook et al., supra; Ausubel et al., supra).

If necessary, the inclusion bodies are solubilized, and the lysed cell suspension is typically centrifuged to remove unwanted insoluble matter. Proteins that formed the inclusion bodies may be renatured by dilution or dialysis with a compatible buffer. Suitable solvents include, but are not limited to urea (from about 4 M to about 8 M), formamide (at least about 80%, volume/volume basis), and guanidine hydrochloride (from about 4 M to about 8 M). Some solvents which are capable of solubilizing aggregate-forming proteins, for example SDS (sodium dodecyl sulfate), 70% formic acid, are inappropriate for use in this procedure due to the possibility of irreversible denaturation of the proteins, accompanied by a lack of immunogenicity and/or activity. Although guanidine hydrochloride and similar agents are denaturants, this denaturation is not irreversible and renaturation may occur upon removal (by dialysis, for example) or dilution of the denaturant, allowing re-formation of immunologically and/or biologically active protein. Other suitable buffers are known to those skilled in the art. The protein of choice is separated from other bacterial proteins by standard separation techniques, e.g., ion exchange chromatography, ammonium sulfate fractionation, etc.

### B. Standard Protein Separation Techniques for Purifying Proteins of the Invention

### (1) Solubility Fractionation

Often as an initial step, particularly if the protein mixture is complex, an initial salt fractionation can separate many of the unwanted host cell proteins (or proteins derived from the cell culture media) from the recombinant protein of interest. The preferred salt is ammonium sulfate. Ammonium sulfate precipitates proteins by effectively reducing the amount of water in the protein mixture. Proteins then precipitate on the basis of their solubility. The more hydrophobic a protein is, the more likely it is to precipitate at lower ammonium sulfate concentrations. A typical protocol includes adding saturated ammonium sulfate to a protein solution so that the resultant ammonium sulfate concentration is between 20-30%. This concentration will precipitate the most hydrophobic of proteins. The precipitate is then discarded (unless the protein of interest is hydrophobic) and ammonium sulfate is added to the supernatant to a concentration known to precipitate the protein of interest. Alternatively, the protein of interest in the supernatant can be further purified using standard protein purification techniques. The precipitate is then solubilized in buffer and the excess salt removed if necessary, either through dialysis or diafiltration. Other methods that rely on solubility of proteins, such as cold ethanol precipitation, are well known to those of skill in the art and can be used to fractionate complex protein mixtures.

### (2) Size Differential Filtration

The molecular weight of the protein, e.g., PA-U1, etc., can be used to isolated the protein from proteins of greater and lesser size using ultrafiltration through membranes of different pore size (for example, Amicon or Millipore membranes). As a first step, the protein mixture is ultrafiltered through a membrane with a pore size that has a lower molecular weight cut-off than the molecular weight of the protein of interest. The retentate of the ultrafiltration is then ultrafiltered against a membrane with a molecular cut off greater than the molecular weight of the protein of interest. The recombinant protein will pass through the membrane into the filtrate. The filtrate can then be chromatographed as described below.

(3) Column chromatography

The protein of choice can also be separated from other proteins on the basis of its size, net surface charge, hydrophobicity, and affinity for ligands. In addition, antibodies raised against proteins can be conjugated to column matrices and the proteins immunopurified. All of these methods are well known in the art. It will be apparent to one of skill that chromatographic techniques can be performed at any scale and using equipment from many different manufacturers (e.g., Pharmacia Biotech).

In some embodiments, the proteins are purified from culture supernatants of Bacillus. Briefly, the proteins are purified by making a culture supernatant 5 mM in EDTA, 35% saturated in ammonium sulfate and 1% in phenyl-Sepharose Fast Flow (Pharmacia). The phenyl-Sepharose Fast Flow is then agitated and collected. The collected resin is washed with 35% saturated ammonium sulfate and the protective antigens were then eluted with 10 mM HEPES-1 mM EDTA (pH 7.5). The proteins can then be further purified using a MonoQ column (Pharmacia Biotech). The proteins can be eluted using a NaCl gradient in 10 mM CHES (2-[N-cyclohexylamino]ethanesulfonic acid)-0.06% (vol/vol) ethanolamine (pH 9.1). The pooled MonoQ fractions can then be dialyzed against the buffer of choice for subsequent analysis or applications.

### VII. Assays for Measuring Changes in Cell Growth and Angiogenesis

The administration of a functional PA and LF combination of the invention to a cell can inhibit cellular proliferation of certain cell types that overexpress MMPs and proteins of the plasminogen activation system, e.g., cancer cells, cells involved in inflammation, stimulated endothelial cells and the like. One of skill in the art can readily identify functional proteins and cells using methods that are well known in the art. Changes in cell growth can be assessed by using a variety of in vitro and in vivo assays, e.g., MTT assay, ability to grow on soft agar, changes in contact inhibition and density limitation of growth, changes in growth factor or serum dependence, changes in the level of tumor specific markers, changes in invasiveness into Matrigel, changes in cell cycle pattern, changes in tumor growth in vivo, such as in normal and transgenic mice, etc.

### A. Assays for Changes in Cell Growth by Administration of Protective Antigen and Lethal Factor

One or more of the following assays can be used to identify proteins of the invention which are capable of regulating cell proliferation. The phrase “protective antigen constructs” refers to a protective antigen protein of the invention. Functional protective antigen constructs identified by the following assays can then be used to treat disease and conditions, e.g., to inhibit abnormal cellular proliferation and transformation. Thus, these assays can be used to identify protective antigen proteins that are useful in conjunction with lethal factor containing proteins to inhibit cell growth of tumors, cancers, cancerous cells, and other pathogenic cell types.

### (1) Soft Agar Growth or Colony Formation in Suspension

Soft agar growth or colony formation in suspension assays can be used to identify protective antigen constructs, which when used in conjunction with a LF construct, inhibit abnormal cellular proliferation and transformation. Typically, transformed host cells (e.g., cells that grow on soft agar) are used in this assay. Techniques for soft agar growth or colony formation in suspension assays are described in Freshney, Culture of Animal Cells a Manual of Basic Technique, 3rd ed., Wiley-Liss, New York (1994), herein incorporated by reference. See also, the methods section of Garkavtsev et al. (1996), supra, herein incorporated by reference.

Normal cells require a solid substrate to attach and grow. When the cells are transformed, they lose this phenotype and grow detached from the substrate. For example, transformed cells can grow in stirred suspension culture or suspended in semi-solid media, such as semi-solid or soft agar. The transformed cells, when transfected with tumor suppressor genes, regenerate normal phenotype and require a solid substrate to attach and grow.

Administration of an active protective antigen protein and an active LF containing protein to transformed cells would reduce or eliminate the host cells' ability to grow in stirred suspension culture or suspended in semi-solid media, such as semi-solid or soft. This is because the transformed cells would regenerate anchorage dependence of normal cells, and therefore require a solid substrate to grow. Therefore, this assay can be used to identify protective antigen constructs that can function with a lethal factor protein to inhibit cell growth. Once identified, such protective antigen constructs can be used in a number of diagnostic or therapeutic methods, e.g., in cancer therapy to inhibit abnormal cellular proliferation and transformation.

### (2) Contact Inhibition and Density Limitation of Growth

Contact inhibition and density limitation of growth assays can be used to identify protective antigen constructs which are capable of inhibiting abnormal proliferation and transformation in host cells. Typically, transformed host cells (e.g., cells that are not contact inhibited) are used in this assay. Administration of a protective antigen construct and a lethal factor construct to these transformed host cells would result in cells which are contact inhibited and grow to a lower saturation density than the transformed cells. Therefore, this assay can be used to identify protective antigen constructs which are useful in compositions for inhibiting cell growth. Once identified, such protective antigen constructs can be used in disease therapy to inhibit abnormal cellular proliferation and transformation.

Alternatively, labeling index with [³H]-thymidine at saturation density can be used to measure density limitation of growth. See Freshney (1994), supra. The transformed cells, when treated with a functional PA/LF combination, regenerate a normal phenotype and become contact inhibited and would grow to a lower density. In this assay, labeling index with [³H]-thymidine at saturation density is a preferred method of measuring density limitation of growth. Transformed host cells are treated with a protective antigen construct and a lethal factor construct (e.g., LP59) and are grown for 24 hours at saturation density in non-limiting medium conditions. The percentage of cells labeling with [³H]-thymidine is determined autoradiographically. See, Freshney (1994), supra. The host cells treated with a functional protective antigen construct would give arise to a lower labeling index compared to control (e.g., transformed host cells treated with a non-functional protective antigen construct or non-functional lethal factor construct).

### (3) Growth Factor or Serum Dependence

Growth factor or serum dependence can be used as an assay to identify functional protective antigen constructs. Transformed cells have a lower serum dependence than their normal counterparts (see, e.g., Temin, J. Natl. Cancer Insti. 37:167-175 (1966); Eagle et al., J. Exp. Med. 131:836-879 (1970)); Freshney, supra. This is in part due to release of various growth factors by the transformed cells. When a tumor suppressor gene is transfected and expressed in these transformed cells, the cells would reacquire serum dependence and would release growth factors at a lower level. Therefore, this assay can be used to identify protective antigen constructs which are able to act in conjunction with a lethal factor to inhibit cell growth. Growth factor or serum dependence of transformed host cells which are transfected with a protective antigen construct can be compared with that of control (e.g., transformed host cells which are treated with a non-functional protective antigen or non-functional lethal factor). Transformed host cells treated with a functional protective antigen would exhibit an increase in growth factor and serum dependence compared to control.

### B. Assays for Changes in Angiogenesis and Endothelial Migration by Administration of Protective Antigen and Lethal Factor

### 1. Direct Measurement of Proliferation of Endothelial Cells

Any of a number well known methods to measure cell proliferation can be adapted for use in monitoring the proliferation of endothelial cells during angiogenesis. These include measurement of the incorporation of labeled DNA precursors such as ³H-thymidine and BrdU or through the measurement of cell markers that are expressed in proliferating cells, such PCNA (see, e.g., Goldsworthy et al. Envir. Health Pros. 101:59-66 (1993).

### 2. Cell Migration Assays

There are several tests that can be used to determine the migratory response of endothelial cells during angiogenesis (see, e.g., Schor et al. In: Murray, J. C., ed. Angiogenesis protocols Totowa, N.J.: Humana Press, 163-204 (2001). Many such methods employ blind-well chemotaxis chambers in which endothelial cells are place on the upper layer of a cell-permeable filter and endothelial cells are permitted to migrate in response to a test angiogenic factor placed in the medium below the filter. Quantitation entails enumeration of retained cells versus those that have migrated across the filter.

### 3. Tube Formation

Tube formation assays measure the ability of endothelial cells to form three-dimensional structures tubular structures as part of the angiogenic process (see, e.g., Madri et al. J. Cell Biol. 106:1375-84 (1988)). Endothelial cells have been shown to form tubules spontaneously after sufficient time to lay down extracellular matrix components. Tube formation can be enhanced in vitro through the use of collagen or fibrin clots to coat plastic culture dishes. Tube formation assays have been facilitated by the use of Matrigel (a matrix-rich product prepared from Engelbreth-Holm-Swarm (EHS) tumor cells, whose primary component is laminin). Matrigel allows the formation of tubes within 24 hours of plating (see, e.g., Grant et al. J. Cell Physiol. 153:614-25 (1992)).

### 4. Organ Culture Assays

In the rat aortic ring assay, isolated rat aorta is cut into segments that are placed in culture, generally in a matrix-containing environment such as Matrigel (see, e.g., Nicosia et al., Lab Invest. 63:115-122 (1990). Over the next 7-14 days, the explants are monitored for the outgrowth of endothelial cells. Quantitation is achieved by measurement of the length and abundance of vessel-like extensions from the explant. Use of endothelium-selective reagents such as fluorescein-labeled BSL-I allows quantitation by pixel counts.

A variation of the rat aortic ring assay is the chick aortic arch assay which entails the dissection of aortic arches from 12-14 day chick embryos which are cut into rings similar to those used in the rat aortic ring assay. When the rings are placed on Matrigel, substantial outgrowth of cells occurs within 48 hours, with the formation of vessel-like structures readily apparent (see, e.g., Muthukkaruppan et al. Proc. Am. Assoc. Cancer Res. 41:65 (2000)). If the aortic arch is everted before plating, the time can be reduced to 24 hours, thus, allowing an assay time of 1-3 days.

Quantitation of both assays can be achieved by use of fluorescein-labeled lectins such as BSL-I and BSL-B4 or by staining of the cultures with labeled antibodies to CD31, combined with standard imaging techniques.

### 5. In Vivo Assays

A number of in vivo assay systems have been developed including the chick chorioallantoic membrane (CAM) assay, an in vivo Matrigel plug assay, and a group of assays that use implants of sponges containing test cells or substances.

In one form of the CAM assay, the chorioallantoic membrane (CAM) of 7-9 day chick embryos was exposed by making a window in the egg shell, and tissue or organ grafts were then placed directly on the CAM. The window was sealed, eggs were reincubated, and the grafts were recovered after an appropriate length of incubation time. The grafts are then scored for growth and vascularization (see, e.g., Brooks et al. Methods Mol. Biol. 129:257-269 (1999)). A modification of this technique involves transferring the entire contents of an egg onto a plastic culture dish.

In the corneal angiogenesis assay, a test pocket is made in the cornea of rabbit or mice eyes, and test tumors or tissues, when introduced into the pocket, elicit the ingrowth of new vessels from the peripheral limbal vasculature (see e.g., Gimbrone et al. J. Exp. Med. 136:261-276 (1974); Muthukkaruppan et al. Science 205:1416-1418 (1979)). Slow release materials such as ELVAX (ethylene vinyl copolymer) or Hydron can be used to introduce test substances into the corneal pocket. Alternatively, sponge material may be used test substances. The angiogenic response can be directly observed or else fluorochrome-labeled high-molecular weight dextran can be injected into the mouse or rabbit corneal vasculature.

The Matrigel plug assay involves the subcutaneous injection of Matrigel containing test cells or substances, where upon the Matrigel solidifies to form a plug. The plug is then recovered after 7-21 days in the animal and examined histologically to determine the extent to which blood vessels have entered it (see, e.g., Passaniti et al. Lab Invest. 67:519-528 (1982)). A variety of methods can be used to quantitate blood vessel formation, including fluorescence measurement of plasma volume using FITC-labeled dextran 150, or by measuring the amount of hemoglobin present in the plug.

### VIII. Tumor Specific Markers Levels

Tumor cells release an increased amount of certain factors (hereinafter “tumor specific markers”) than their normal counterparts. F or example, tumor angiogenesis factor (TAF) is released at a higher level in tumor cells than their normal counterparts. See, e.g., Folkman, Angiogenesis and cancer, Sem. Cancer Biol. (1992)). Tumor specific markers can be assayed for to identify protective antigen constructs, which when administered with a lethal factor construct, decrease the level of release of these markers from host cells. Typically, transformed or tumorigenic host cells are used. Administration of a protective antigen and a lethal factor to these host cells would reduce or eliminate the release of tumor specific markers from these cells. Therefore, this assay can be used to identify protective antigen constructs are functional in suppressing tumors.

Various techniques which measure the release of these factors are described in Freshney (1994), supra. Also, see, Unkless et al., J. Biol. Chem. 249:4295-4305 (1974); Strickland & Beers, J. Biol. Chem. 251:5694-5702 (1976); Whur et al., Br. J. Cancer 42:305-312 (1980); Gulino, Angiogenesis, tumor vascularization, and potential interference with tumor growth. In Mihich, E. (ed): “Biological Responses in Cancer.” New York, Plenum (1985); Freshney Anticancer Res. 5:111-130 (1985).

### IX. Cytotoxicity Assay with MTT

The cytotoxicity of a particular PA/LF combination can also be assayed using the MTT cytotoxicity assay. Cells are seeded and grown to 80 to 100% confluence. The cells are then were washed twice with serum-free DMEM to remove residual FCS and contacted with a particular PA/LF combination. MTT (3-[4,5-dimethylthiazol-2-yl]-2,5-diphenyltetrazolium bromide) is then added to the cells and oxidized MTT (indicative of a live cell) is solubilized and quantified.

### X. Invasiveness into Matrigel

The degree of invasiveness into Matrigel or some other extracellular matrix constituent can be used as an assay to identify protective antigen constructs which are capable of inhibiting abnormal cell proliferation and tumor growth. Tumor cells exhibit a good correlation between malignancy and invasiveness of cells into Matrigel or some other extracellular matrix constituent. In this assay, tumorigenic cells are typically used. Administration of an active protective antigenllethal factor protein combination to these tumorigenic host cells would decrease their invasiveness. Therefore, functional protective antigen constructs can be identified by measuring changes in the level of invasiveness between the tumorigenic cells before and after the administration of the protective antigen and lethal factor constructs.

Techniques described in Freshney (1994), supra, can be used. Briefly, the level of invasion of tumorigenic cells can be measured by using filters coated with Matrigel or some other extracellular matrix constituent. Penetration into the gel, or through to the distal side of the filter, is rated as invasiveness, and rated histologically by number of cells and distance moved, or by prelabeling the cells with ¹²⁵I and counting the radioactivity on the distal side of the filter or bottom of the dish. See, e.g., Freshney (1984), supra.

### XI. G₀/G₁ Cell Cycle Arrest Analysis

G₀/G₁ cell cycle arrest can be used as an assay to identify functional protective antigen construct. PA/LF construct administration can cause G₁ cell cycle arrest. In this assay, cell lines can be used to screen for functional protective antigen constructs. Cells are treated with a putative protective antigen construct and a lethal factor construct. The cells can be transfected with a nucleic acid comprising a marker gene, such as a gene that encodes green fluorescent protein. Administration of a functional protective antigen/lethal factor combination would cause G₀/G₁ cell cycle arrest. Methods known in the art can be used to measure the degree of G₁ cell cycle arrest. For example, the propidium iodide signal can be used as a measure for DNA content to determine cell cycle profiles on a flow cytometer. The percent of the cells in each cell cycle can be calculated. Cells exposed to a functional protective antigen would exhibit a higher number of cells that are arrested in G₀/G₁ phase compared to control (e.g., treated in the absence of a protective antigen).

### XII. Tumor Growth In Vivo

Effects of PA/LF on cell growth can be tested in transgenic or immune-suppressed mice. Transgenic mice can be made, in which a tumor suppressor is disrupted (knock-out mice) or a tumor promoting gene is overexpressed. Such mice can be used to study effects of protective antigen as a method of inhibiting tumors in vivo.

Knock-out transgenic mice can be made by insertion of a marker gene or other heterologous gene into a tumor suppressor gene site in the mouse genome via homologous recombination. Such mice can also be made by substituting the endogenous tumor suppressor with a mutated version of the tumor suppressor gene, or by mutating the endogenous tumor suppressor, e.g., by exposure to carcinogens.

A DNA construct is introduced into the nuclei of embryonic stem cells. Cells containing the newly engineered genetic lesion are injected into a host mouse embryo, which is re-implanted into a recipient female. Some of these embryos develop into chimeric mice that possess germ cells partially derived from the mutant cell line. Therefore, by breeding the chimeric mice it is possible to obtain a new line of mice containing the introduced genetic lesion (see, e.g., Capecchi et al., Science 244:1288 (1989)). Chimeric targeted mice can be derived according to Hogan et al., Manipulating the Mouse Embryo: A Laboratory Manual, Cold Spring Harbor Laboratory (1988) and Teratocarcinomas and Embryonic Stem Cells: A Practical Approach, Robertson, ed., IRL Press, Washington, D.C. (1987).

These knock-out mice can be used as hosts to test the effects of various protective antigen constructs on cell growth. These transgenic mice with a tumor suppressor gene knocked out would develop abnormal cell proliferation and tumor growth. They can be used as hosts to test the effects of various protective antigen constructs on cell growth. For example, introduction of protective antigen constructs and lethal factor constructs into these knock-out mice would inhibit abnormal cellular proliferation and suppress tumor growth.

Alternatively, various immune-suppressed or immune-deficient host animals can be used. For example, genetically athymic “nude” mouse (see, e.g., Giovanella et al., J. Natl. Cancer Inst. 52:921 (1974)), a SCID mouse, a thymectomized mouse, or an irradiated mouse (see, e.g., Bradley et al., Br. J. Cancer 38:263 (1978); Selby et al., Br. J. Cancer 4152 (1980)) can be used as a host. Transplantable tumor cells (typically about 10⁶ cells) injected into isogenic hosts will produce invasive tumors in a high proportions of cases, while normal cells of similar origin will not. In hosts which developed invasive tumors, cells are exposed to a protective antigen construct/lethal factor combination (e.g., by subcutaneous injection). After a suitable length of time, preferably 4-8 weeks, tumor growth is measured (e.g., by volume or by its two largest dimensions) and compared to the control. Tumors that have statistically significant reduction (using, e.g., Student's T test) are said to have inhibited growth. Using reduction of tumor size as an assay, functional protective antigen constructs which are capable of inhibiting abnormal cell proliferation can be identified. This model can also be used to identify functional mutant versions of protective antigen.

### XIII. Pharmaceutical Compositions Administration

Protective antigen containing proteins and lethal factor containing proteins can be administered directly to the patient, e.g., for inhibition of cancer, tumor, or precancer cells in vivo, etc. Administration is by any of the routes normally used for introducing a compound into ultimate contact with the tissue to be treated. The compounds are administered in any suitable manner, preferably with pharmaceutically acceptable carriers. Suitable methods of administering such compounds are available and well known to those of skill in the art, and, although more than one route can be used to administer a particular composition, a particular route can often provide a more immediate and more effective reaction than another route.

Pharmaceutically acceptable carriers are determined in part by the particular composition being administered, as well as by the particular method used to administer the composition. Accordingly, there is a wide variety of suitable formulations of pharmaceutical compositions of the present invention (see, e.g., Remington's Pharmaceutical Sciences, 17th ed. 1985)). For example, if in vivo delivery of a biologically active protective antigen protein is desired, the methods described in Schwarze et al. (see, Science 285:1569-1572 (1999)) can be used.

The compounds, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen, and the like.

Formulations suitable for parenteral administration, such as, for example, by intravenous, intramuscular, intradermal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. In the practice of this invention, compositions can be administered, for example, by intravenous infusion, orally, topically, intraperitoneally, intravesically or intrathecally. The formulations of compounds can be presented in unit-dose or multi-dose sealed containers, such as ampules and vials. Injection solutions and suspensions can be prepared from sterile powders, granules, and tablets of the kind previously described.

The dose administered to a patient (“a therapeutically effective amount”), in the context of the present invention should be sufficient to effect a beneficial therapeutic response in the patient over time. The dose will be determined by the efficacy of the particular compound employed and the condition of the patient, as well as the body weight or surface area of the patient to be treated. The size of the dose also will be determined by the existence, nature, and extent or any adverse side-effects that accompany the administration of a particular compound or vector in a particular patient.

In determining the effective amount of the compound(s) to be administered in the treatment or prophylaxis of cancer, the physician evaluates circulating plasma levels of the respective compound(s), progression of the disease, and the production of anti-compound antibodies. In general, the dose equivalent of a compound is from about 1 ng/kg to 10 mg/kg for a typical patient. Administration of compounds is well known to those of skill in the art (see, e.g., Bansinath et al., Neurochem. Res. 18:1063-1066 (1993); Iwasaki et al., Jpn. J. Cancer Res. 88:861-866 (1997); Tabrizi-Rad et al., Br. J. Pharmacol. 111:394-396 (1994)).

For administration, compounds of the present invention can be administered at a rate determined by the LD-50 of the particular compound, and its side-effects at various concentrations, as applied to the mass and overall health of the patient. Administration can be accomplished via single or divided doses.

### EXAMPLES

The following examples are offered to illustrate, but not to limit, the claimed invention.

### Introduction

The Sanger Institute's Cancer Genome Project and subsequent studies conducted by other investigators have identified the BRAF V600E mutation as occurring in approximately 70% of human melanomas and less frequently in other cancer types, such as colon, ovarian, and papillary thyroid cancer, representing about 8% of total human cancers (Davies, H. et al., Nature, 417, 949-954 (2002); Sebolt-Leopold, J. S, and Herrera, R., Nat. Rev. Cancer, 4, 937-947 (2004)). BRAF is immediately downstream of RAS in the kinase cascade and there is a trend showing that the BRAF mutation is present in cancer types with activating RAS mutations (Davies, H. et al., Nature, 417:949-954 (2002); Sebolt-Leopold, J. S, and Herrera, R., Nat. Rev. Cancer, 4:937-947 (2004)). However, the RAS and the BRAF mutations typically demonstrate mutual exclusivity, suggesting that either mutation is sufficient to deregulate the common downstream MEK-ERK kinase cascade, upon which the tumors with these mutations are dependent for survival.

Recently, based on their NCI60 anticancer drug screen, Rosen and colleagues demonstrated that tumor cells with the BRAF, but not the RAS mutation, are sensitive to MEK inhibition (Solit, D. B. et al., Nature (2005)). It is not surprising that tumors with activating RAS mutations are less sensitive to MEK inhibition, because RAS can also activate the PI3K pathway to support tumor survival (Curtin, J. A. et al., N. Engl. J. Med., 353:2135-2147 (2005)). Therefore, molecular targeting of the BRAF-MEK-ERK pathway would be selective to tumors with the BRAF mutation. We reported recently that LT, which can inactivate MEK1/2 and other MEKs by enzymatic cleavage, is selectively toxic to human melanoma cell lines having the BRAF mutation, but not to those with RAS mutations (Abi-Habib, R. J. et al., Mol. Cancer. Ther., 4:1303-1310 (2005)). This LT selective toxicity to human melanomas with BRAF V600E was verified in an experimental therapy of SK-MEL-28 melanoma xenografts in athymic mice (Abi-Habib, R. J. et al., Clin. Cancer Res., 12, 7437-7443 (2006)). However, the fact that anthrax LT is an important virulence factor in anthrax pathogenesis and has recognized toxicity to mice (Moayeri, M. et al., J. Clin. Invest., 112:670-682 (2003)) means that wild-type LT might not be accepted for use in human patients.

To achieve the goal of decreasing in vivo toxicity of LT while retaining its anti-tumor activity, we previously developed an attenuated version of the toxin (PA-L1/LF), which cannot be cleaved by the ubiquitously expressed protease furin, but is instead activated by MMPs, including MMP-2, MMP-9, and MT1-MMP (membrane type 1 MMP). MMPs are involved in tumor survival, angiogenesis, invasive growth, and metastasis (Liu, S. et al., Cancer Res., 60:6061-6067 (2000); Liu, S. et al., Nat. Biotechnol., 23:725-730 (2005)). We showed that all the cancer cells tested express MMPs and thus, are highly sensitive to PA-L1/FP59. Furthermore, the cancer cells with the BRAF mutation are susceptible to both PA/LF and PAL1/LF to comparable degrees, whereas the cancer cells without BRAF V600E are generally resistant to the toxins. Moreover, in addition to melanoma cells, colon cancer cells with the BRAF mutation are also sensitive to the toxins, indicating that the addiction to the activating BRAF mutation is not cell lineage-specific. We found that PA-L1/LF has much lower toxicity than wild-type toxin in the mice; C57BL/6 mice easily tolerate 6 doses of 45/15 μg of PA-L1/LF given systemically, while they can only tolerate doses close to 15/5 μg of PA/LF, and cannot tolerate even 2 doses of 30/10 μg of PA/LF (Example 2, Table 1). These results indicate that most of the normal tissues lack expression of MMPs and that PA-L1/LF is much safer than PA/LF when used in vivo.

A first surprising finding in the work described herein came from an in vivo anti-tumor efficacy study. We found that PA-L1/LF has a potent anti-tumor activity not only against human melanomas with BRAF V600E, but also against other human tumor types, including colon and lung carcinomas, and mouse tumors, regardless of their BRAF status (Example 3). We further observed that this potent generic anti-tumor activity is due largely to targeting of tumor vasculature and angiogenic processes. A key role for angiogenesis was evident from data showing that: (a) LT significantly down-regulates IL8 expression in all the four cancer cells tested (IL8 is a strong pro-inflammatory mediator involved in tumor angiogenesis); (b) tumor blood vessels are largely absent in A549/ATCC tumors treated with PA-L1/LF in comparison with those treated with PBS; (c) PA-L1/LF strongly inhibits the migration of human primary endothelial cells towards a gradient of serum and angiogenic factors, an essential step for tumor angiogenesis; (d) anthrax toxin-receptor-deficient CHO tumor xenografts are susceptible to PA-L1/LF; and most importantly, (e) PA-L1/LF can efficiently block angiogenesis in vivo. See Examples 3-6 below.

Recently, Sparmann and Bar-Sagi showed that activation of RAS in human cancer cells results in up-regulation of IL8, leading to recruitment of mouse neutrophils and macrophages, which in turn produce growth factors and angiogenic factors to promote tumor angiogenesis and growth (Sparmann, A. and Bar-Sagi, D., Cancer Cell, 6:447-458 (2004)). They further showed that inhibition of IL8 by a neutralizing antibody or ablation of macrophages can significantly inhibit the growth of tumor xenografts (Sparmann, A. and Bar-Sagi, D., Cancer Cell, 6:447-458 (2004)), attesting to the importance of IL8 and macrophages in tumorigenesis. To determine whether the anti-tumor efficacy of PA-L1/LF was solely due to its ability to down-regulate expression of IL8, we transfected IL8 lacking 3′ UTR into A549/ATCC and C32 cells; we found these tumor xenografts with ‘resistant’ IL8 are still very susceptible to PA-L1/LF. See Example 5.

It has been noted for two decades that the macrophages from certain inbred mice and rats are uniquely sensitive to LT in that these macrophages can be lysed by the toxin in just 90 minutes (Friedlander, 1986). Recently, the genetic trait of the sensitivity has been assigned to the Nalplb locus, encoding a polymorphic protein existing in the inflammasome complex (Boyden, E. D. and Dietrich, W. F., Nat. Genet., 38:240-244 (2006)). How Nalplb is linked to macrophage sensitivity to LT is still unclear. We ruled out the possibility that the potent anti-tumor efficacy of PA-L1/LF is due to the unique toxicity of the toxin to tumor associated macrophages because macrophages isolated from the bone marrow of mice used for tumor xenografts are ‘resistant’ to LT. While macrophages derived from BALB/c mice are lysed by PA/LF(LT) within 4 h, macrophages from C57BL6 and nude mice cannot be killed even after 24 h.

Another unexpected finding in the present work is that PA-L1/LF not only displays much lower in vivo toxicity but also shows higher anti-tumor efficacy than does the wild-type toxin. This is due in part to the unexpected greater bioavailability and longer half-life of PA-L1 in circulation as compared to PA. See Example 3. We previously showed that following binding to its cellular receptors, PA must be proteolytically cleaved on cell surfaces for formation and internalization of the PA heptamer into the endocytic pathway (Liu, S. and Leppla, S. H., Mol. Cell, 12:603-613 (2003)). Thus, the rates of processing on cell surfaces are believed to largely determine the clearance of PA proteins from circulation (Moayeri, M. et al., Infect. Immun., 75, 5175-5184 (2007)). The fact that furin protease is widely expressed whereas MMPs are restricted to a small number of normal cells explains why PA-L1 has a longer plasma half-life.

CI-1040 is the first small molecule MEK inhibitor exhibiting anti-tumor activity in vivo, and it has advanced to Phase I and Phase II clinical trials (Sebolt-Leopold, J. S. and

Herrera, R., Nat. Rev. Cancer, 4:937-947 (2004)). However, because of its poor metabolic stability and lack of efficacy in the Phase II trials, further development of this agent was terminated. PD0325901, which is highly similar in structure to CI-1040, belongs to the second generation of MEK inhibitors. This compound, with an IC₅₀ of 1 nM for MEK1/2 inhibition in cells, shows a much higher potency than C-1040 in vivo, demonstrating anti-tumor efficacy to several human tumor xenografts (Sebolt-Leopold, J. S. and Herrera, R., Nat. Rev. Cancer, 4:937-947 (2004)).

Rosen and colleagues further demonstrated that PD0325901 is efficient in inhibition of the growth of human tumor xenografts containing the BRAF V600E, but has limited efficacy against tumors without the BRAF mutation (Solit, D. B. et al., Nature (2005)), indicating that the action of the compound is through direct targeting of the cancer cells. Because of its catalytic nature, LF might be more potent than small molecule MEK inhibitors in targeting the MEK-ERK pathway. LF, at a concentration of only 0.07 nM (6.4 ng/ml), can proteolytically inactivate the majority of MEK1 in CHO cells after incubation with the cells for 90 minutes (Liu, S. et al., Expert Opin. Biol. Ther., 3:843-853 (2003)). As presented previously, LF has an additional advantage over small molecule inhibitors in that it can be specifically delivered to cancer cells using tumor-selective PA proteins (Liu, S. et al., J. Biol. Chem., 276:17976-17984 (2001); Liu, S. and Leppla, S. H., Mol. Cell, 12:603-613 (2003)). Furthermore, in addition to targeting the MEK-ERK pathway, LT also has activity against the other major MAPK pathways via enzymatic cleavage of MEK3 and 6 (p38 pathway) and MEK4 and 7 (JNK pathway) (Baldari, C. T. et al., Immunol. (2006)), providing an explanation for our observations that PA-L1/LF has broader anti-tumor activity than PD0325901. However, in addition to the tumors with the BRAF mutation, we have demonstrated that the tumors without the mutation, including those from human as well as mouse origins and even those derived from the toxin-receptors-deficient CHO cells, are all susceptible to PA-L1/LF. See Example 3.

In summary, the Examples below as described herein show that PA-L1/LF has unanticipated broad anti-tumor activity exceeding the wild-type toxin with respect to both safety and efficacy, due to its direct inactivation of the MEKs, indirect inhibition of tumor angiogenesis, lower non-specific targeting of normal tissues that lack MMPs, and extended plasma half-life compared to wild-type toxin. The modified protective antigen also shows a decreased immunogenicity. Accordingly, MMP-activated anthrax lethal toxin represents an attractive new therapy option for cancer patients. While all tumor types are expected to respond to PA-L1/LF therapy as a result of an anti-angiogenic effect, patients with tumors containing the BRAF mutation may derive additional benefits due to the direct toxicity of the toxin to these cancer cells.

Furthermore, the LF therapeutic approaches of the present invention have an additional advantage over small molecule inhibitors in that LF can be specifically delivered to cancer cells using tumor-selective PA proteins (Liu et al., J. Biol. Chem., 276:17976-17984 (2001); Liu et al., Proc. Natl. Acad. Sci. U.S.A., 100: 657-662 (2003); Liu et al., Nature Biotechnol., 23: 725-730 (2005)). Because of its catalytic nature, LF might be more potent than small molecule MEK inhibitors in targeting the MEK-ERK pathway.

### Example 1

### MMP-Activated Anthrax Lethal Toxin is Cytotoxic to Human Cancer Cells with the BRAF V600E Mutation

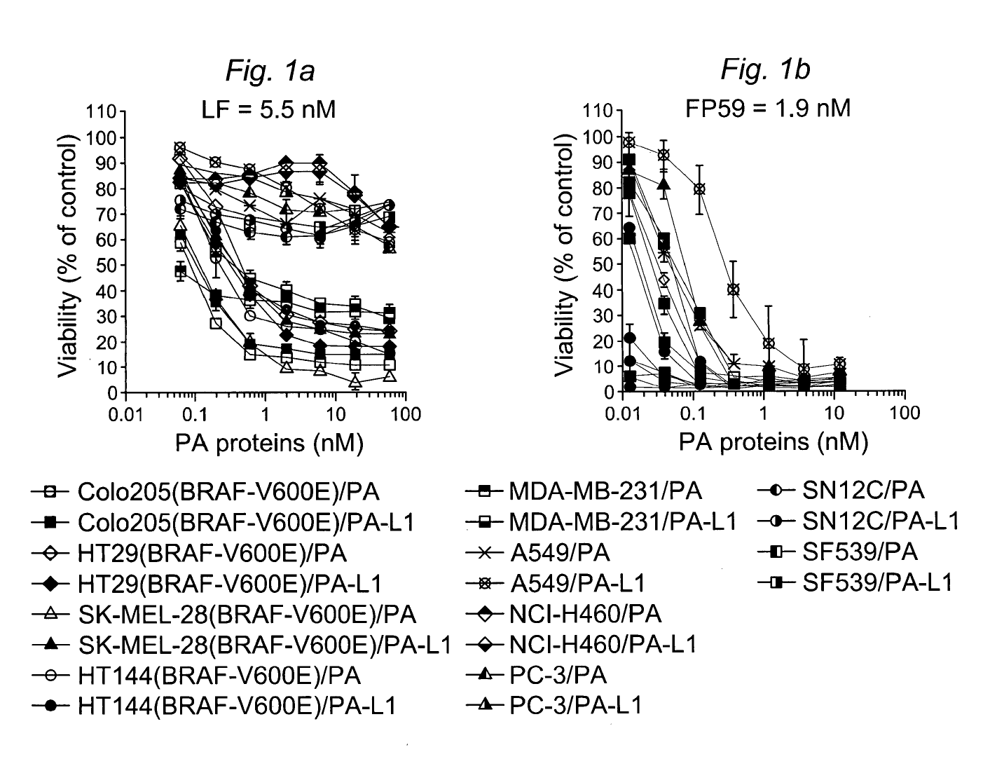

PA-L1 is a mutated PA protein with the furin cleavage site, RKKR, replaced by a MMP-susceptible cleavage sequence, GPLGMLSQ (Liu, S. et al., Cancer Res., 60:6061-6067 (2000)). To evaluate the in vitro anti-tumor activity of the MMP-activated LT (PA-L1/LF), cytotoxicity analyses were performed on four BRAF V600E-containing tumor cell lines from the NCI60 cell set (Shoemaker, R. H., Nat. Rev. Cancer, 6:813-823 (2006)), Colo205 (colon), HT29 (colon), SK-MEL-28 (melanoma), and HT144 (melanoma), in comparison to six BRAF wild type lines, MDA-MB-231 (breast), A594/ATCC (lung), NCI-H460 (lung), PC-3 (prostate), SN12C (renal), and SF539 (central nervous system). We found that PA-L1/LF was cytotoxic to both melanoma and colon cancer cells having the BRAF mutation at potencies comparable to those of wild-type LT (PA/LF) for these cells (FIG. 1A). However, all the tumor cells (except MDA-MB-231) without the BRAF V600E mutation were resistant to both PA/LF and PA-L1/LF (FIG. 1A). These results agree well with the previous findings that the human melanoma cells with the BRAF mutation are sensitive to LT and further extend the conclusion to human colon cancer cells with the BRAF mutation (Abi-Habib, R. J. et al., Mol. Cancer. Ther., 4:1303-1310 (2005)). Thus, not only human melanoma cells but also human colon cancer cells with the BRAF mutation are sensitive to PA/LF and PA-L1/LF.

To exclude the possibility that the general insensitivity of the tumor cells without the BRAF mutation to the anthrax lethal toxins is due to a lack of expression of PA receptors on these cells, the tumor cells were also treated with PA/FP59 and PA-L1/FP59. FP59 is a fusion protein of LF amino acids 1-254 and the catalytic domain of PE (Arora, N. and Leppla, S. H., J. Biol. Chem., 268:3334-3341 (1993)), and can kill any cell type by ADP-ribosylation and, thus, inactivation of EF-2 when it is delivered into the cytosol of the cell in a PA-dependent manner. PA/FP59 and PA-L1/FP59 showed a potent and comparable cytotoxicity to all the human cancer cells tested (FIG. 1B) regardless of their BRAF status, demonstrating that these tumor cells express PA receptors and MMPs. These findings argue that MMP-activated LT may be a useful reagent for tumor targeting.

### Example 2

### Attenuated In Vivo Toxicity of the MMP-Activated Anthrax Lethal Toxin

We next evaluated the toxicity of PA-L1/LF in vivo. Mice were challenged intraperitoneally (i.p.) with 6 doses (three times a week with two-day intervals for two weeks) of PA/LF or PA-L1/LF. A molar ratio of 3:1 of PA protein to LF was used in the challenge experiments based on the fact that each PA heptamer can bind and deliver up to three molecules of LF into cells (Mogridge, J. et al., Proc. Natl. Acad. Sci. U.S.A., 99:7045-7048 (2002)). C57BL/6 mice could tolerate 6 doses of 10/3.3 μg of PA/LF, but could not tolerate doses beyond 15/5 μg of PA/LF. One of 10 mice died after 6 doses of 15/5 μg of PA/LF; and 11 of 11 died after 2 doses of 30/10 μg of PA/LF (Table 1). Several major organ damages associated with vascular collapse had been identified as major lesions in LT-treated mice (Moayeri et al., J. Clin. Investing., 112, 670-682 (2003). In contrast, the mice tolerated as many as 6 doses of 45/15 μg of PA-L1/LF. All the mice survived challenge with 6 doses of 30/10 μg and 45/15 μg of PA-L1/LF, respectively, and lacked any outward sign of toxicity (Table 1). Full necropsy analyses of the C57BL/6 mice treated with 6 doses of 45/15 μg of PA-L1/LF did not reveal any gross abnormalities. Further, extensive histological analyses did not uncover damage in major organs and tissues, including brain, lung, heart, liver, small and large intestines, kidney and adrenal gland, stomach, pancreas, spleen, thyroid, bladder, esophagus, skeletal muscle, thymus, and lymph nodes (data not shown). The sensitivity of the mice to LT varies with genetic background (Moayeri, M. et al., Infect. Immun., 72:4439-4447 (2004)). For instance, BALB/c mice are more sensitive to LT. We found, however, that BALB/c mice could also tolerate 6 doses of 45/15 μg of PA-L1/LF. These results demonstrate that the MMP-activated LT has much lower in vivo toxicity than wild-type toxin; the MTD6 (the maximum tolerated 6 doses) for PA-L1/LF is ≧45/15 μg, whereas that of PA/LF is ≧10/3.3 and <15/5 μg.

TABLE 1
In vivo toxicity of anthrax lethal toxins to mice

| | | Percent survival for 6 doses | | |
|---|---|---|---|---|
| Toxin | Dose | C57BL/6 | BALB/c | Nude mice |
| PA/LF | 10/3.3 μg | 1.00% (515) | — | |
| | 15/5 μg | 90% (9/10) | — | 47% (14/30) |
| | 30/10 μg | 0% (0/11) | — | |
| PA-L1/LF | 15/5 μg | — | — | 100% (10/10) |
| | 30/10 μg | 100% (22/22) | — | 100% (42/42) |
| | 45/15 μg | 100% (11/11) | 100% (5/5) | 70% (28/40) |

“—”: not done.

### Example 3

### MMP-Activated Anthrax Lethal Toxin has Potent and Broad Anti-Tumor Activity In Vivo

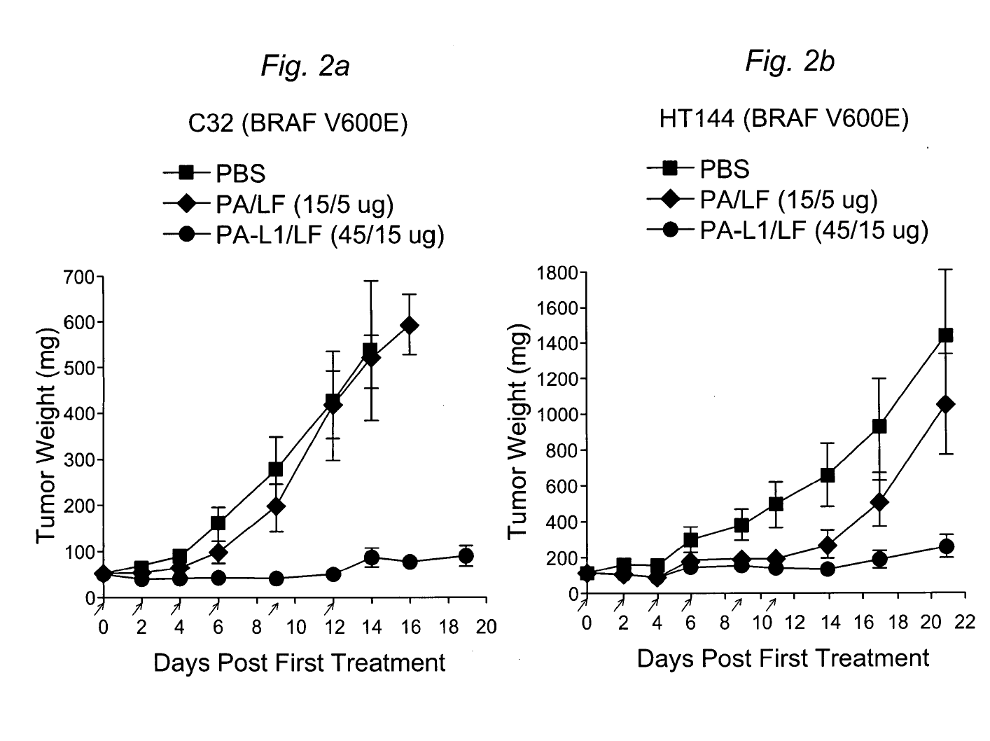

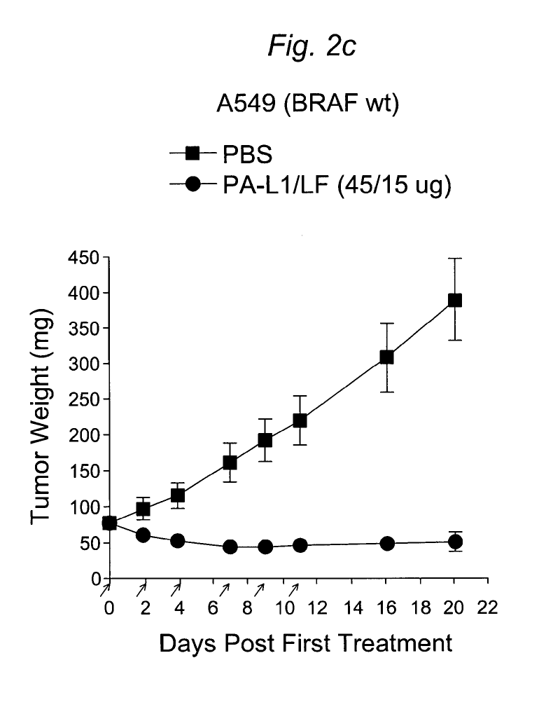

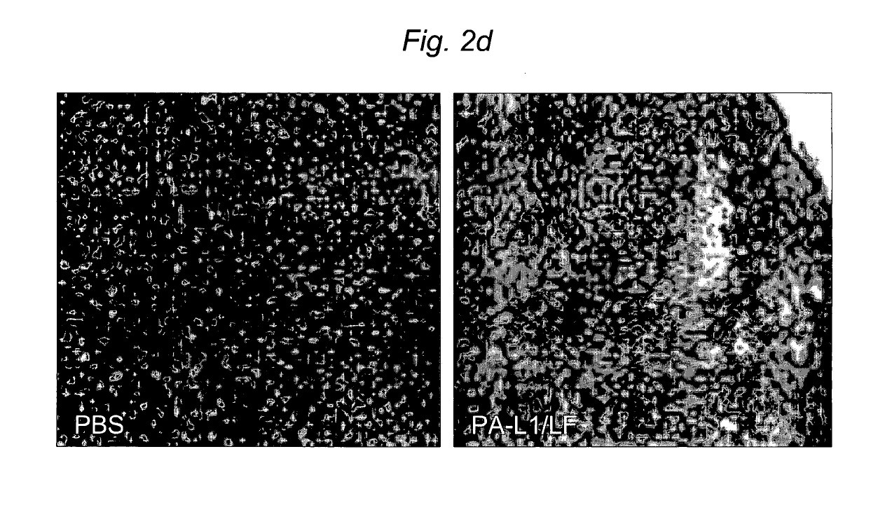

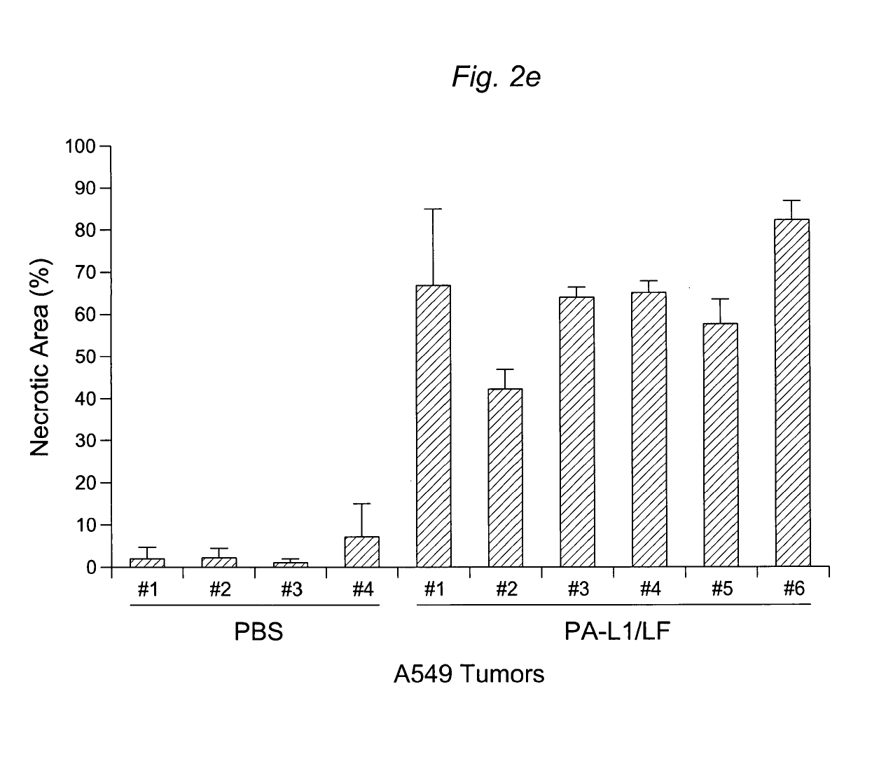

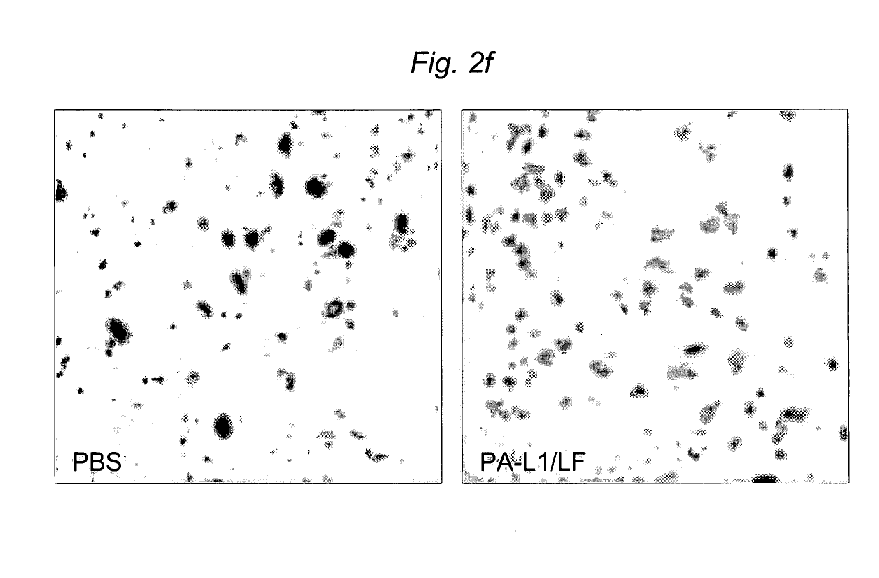

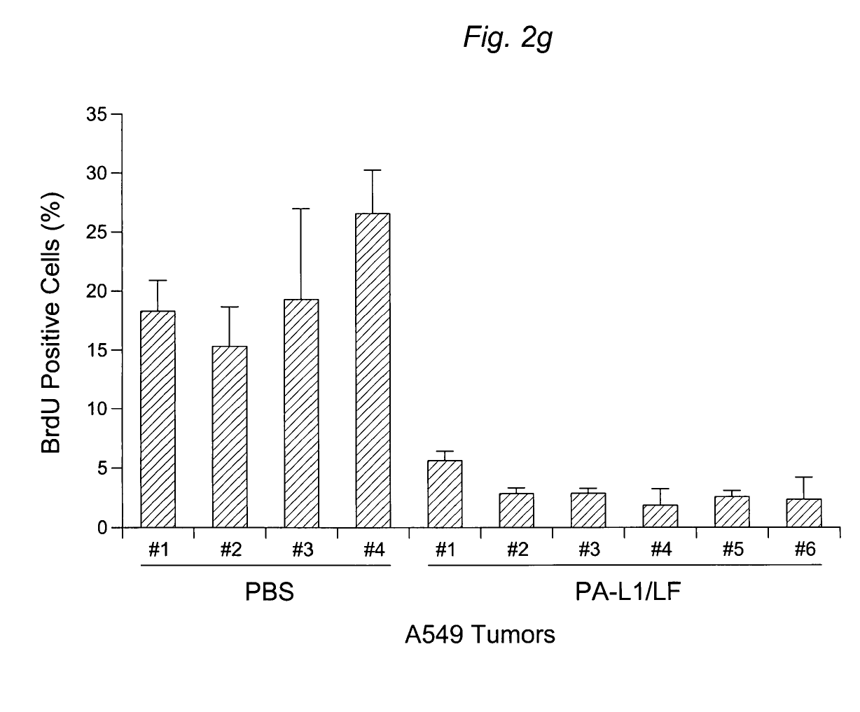

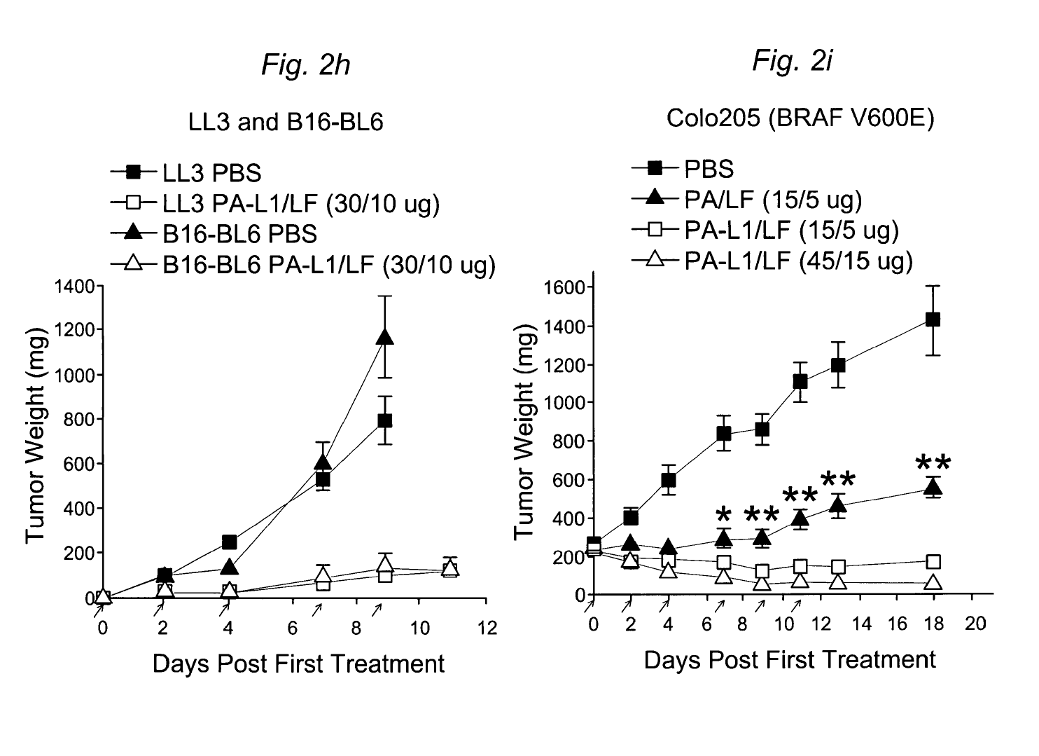

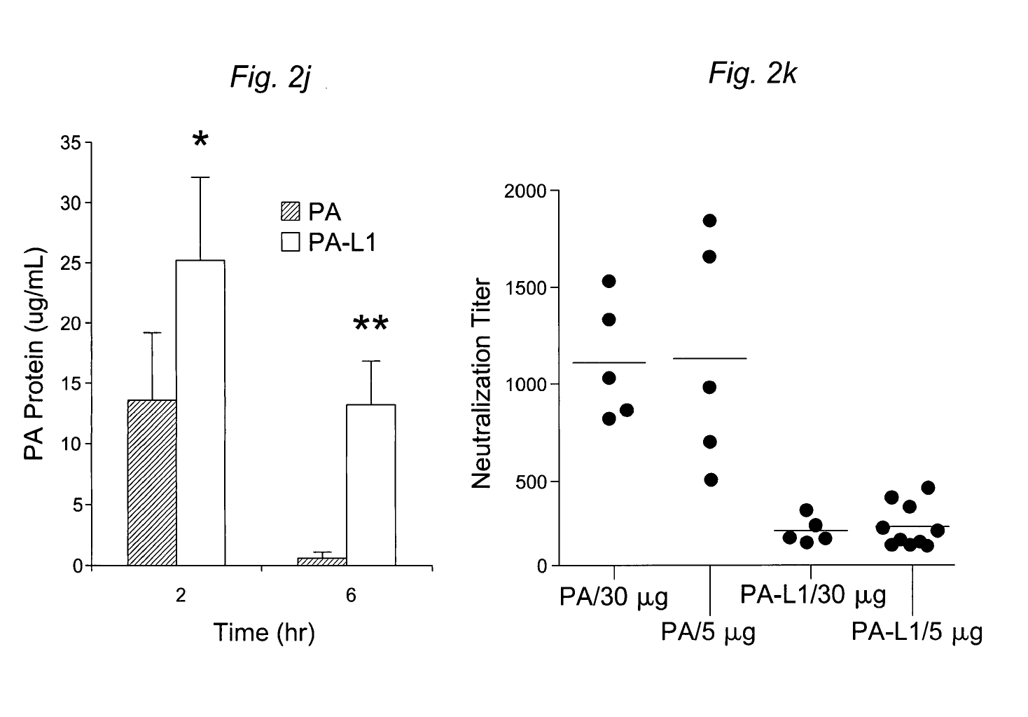

To determine whether the anti-tumor activity of PA-L1/LF in vitro can be recapitulated in vivo, we established human tumor xenografts in nude mice using human melanoma HT144 cells and C32 cells, containing the BRAF V600E mutation, and human non-small cell lung carcinoma A549/ATCC cells, which lack the BRAF mutation. After these tumors were well established, the mice were injected (i.p.) with 6 doses of 45/15 μg of PA-L1/LF (MTD6), 6 doses of 15/5 μg of PA/LF (≈MTD6), or PBS. Remarkably, the two human melanomas with the BRAF mutation were very sensitive to PA-L1/LF, with average tumor sizes just 16% and 17%, respectively, of the control tumors treated with PBS at the time when the control mice required euthanasia due to tumor ulceration in compliance with institutional guidelines (FIG. 2A and FIG. 2B). In the case of C32 melanomas, 30% of the tumors achieved complete regression. In contrast, we observed little or no response of these tumors to wild-type LT (FIG. 2A and FIG. 2B). Unexpectedly, PA-L1/LF also exhibited strong toxicity to A549/ATCC carcinomas that do not have the BRAF mutation, resulting in the eradication of 50% of the established tumors (FIG. 2C). Histological analyses showed that PA-L1/LF treatment induced extensive tumor necrosis, which did not occur in the PBS-treated tumors (FIG. 2D and FIG. 2E). Furthermore, a bromodeoxyuridine (BrdU) incorporation assay demonstrated that while proliferating cells were evident in the PBS-treated tumors, DNA synthesis in the toxin-treated tumors was greatly inhibited, even in areas with living cancer cells (FIG. 2F and FIG. 2G). These results demonstrate that the MMP-activated LT has potent anti-tumor activity not only to human melanomas with the BRAF mutation, but also to another human tumor type that lacks the BRAF mutation.

We further tested the therapeutic efficacy of PA-L1/LF in two mouse syngeneic tumor models. B16-BL6 melanoma and LL3 Lewis lung carcinoma are two highly malignant mouse tumors, growing and disseminating rapidly when transplanted to syngeneic mice. These two tumors demonstrate a poor response to conventional treatments. C57BL/6 mice bearing B16-BL6 melanomas and LL3 Lewis lung carcinomas were treated (i.p.) with 5 doses of 30/10 μg of PA-L1/LF and PBS (FIG. 2H). These tumors were also highly susceptible to the engineered toxin, with the average sizes of B16-BL6 and LL3 tumors treated with the toxin just 10% and 11%, respectively, of those treated with PBS. Because A549/ATCC carcinomas and B16-BL6 melanomas are resistant to PA-L1/LF in the in vitro cytotoxicity assay (FIG. 1A and data not shown) but sensitive in vivo, the above data strongly suggest that the potent anti-tumor efficacy of the modified LT might be through targeting tumor vasculature and angiogenesis.

As shown above, when used at the similar toxic doses (≈MTD6), PA-L1/LF displayed more potent anti-tumor effect than did PA/LF. Next, we directly compared their therapeutic efficacy at the same doses using human colon cancer Colo205 xenografts in nude mice. The Colo205 tumor-bearing mice were treated with 6 doses of 15/5 μg or 45/15 μg of PA-L1/LF, or 15/5 μg of PA/LF. Notably, PA-L1/LF retained remarkable efficacy even when the dose was reduced to 15/5 μg, whereas the same dose of PA/LF only showed a modest anti-tumor effect on Colo205 tumors, which was significantly lower than that of PA-L1/LF (p<0.01) (FIG. 2I). This result was at first surprising, because PA/LF showed similar or higher toxicity than PAL1/LF in all the cancer cells tested (FIG. 1A). We previously reported that the proteolytic processing and the subsequent oligomerization of PA63 on cell surfaces is essential for the cellular uptake and eventual degradation of PA (Liu, S. and Leppla, S. H., J. Biol. Chem., 278:5227-5234 (2003)). Because 6 doses of 15/5 μg of PA/LF showed unacceptable toxicity to nude mice (Table 1), we did not further evaluate the wild-type LT in mice in further studies directed toward the identification of anti-tumor mechanisms of the MMP-activated LT.

### Example 4

### Higher Bioavailability and Decreased Immunogenicity of the MMP-Activated Protective Antigen

The above results showing that PA-L1/LF has higher in vivo anti-tumor activity than PA/LF (FIG. 2I) were at first surprising, because PA/LF showed similar or higher in vitro toxicity than PA-L1/LF in all the cancer cells tested (FIG. 1A). We previously reported that the proteolytic processing and the subsequent oligomerization of PA63 on cell surfaces are essential for the cellular uptake and eventual degradation of PA in the endocytic pathway (Liu and Leppla, J. Biol. Chem., 278: 5227-5234 (2003)). Given that fewer cell types express MMPs than furin or furin-like proteases, we assumed that PA-L1 might be cleared from plasma more slowly than PA. To test this hypothesis, 100 μg of PA or PA-L1 was intravenously injected into mice, and the plasma clearance of the PA proteins was measured (FIG. 2J). We demonstrated that PA-L1 remained in circulation much longer than PA did; 6 h after the injection, when PA was hardly detected (0.57±0.23 μg/ml), there was still a significant amount of PA-L1 in the plasma (12.9±3.6 μg/ml), indicating that PA-L1 has a better bioavailability in vivo than PA, which may contribute to its higher in vivo anti-tumor activity.

PA has a well-known immunogenic activity and is a major component of the only licensed anthrax vaccine (Anthrax Vaccine Absorbed) currently used in USA. This raises a practical concern that repeat uses of PA proteins in therapy may induce neutralizing antibodies that may interfere with their later uses. The fact that PA-L1 can not be internalized and degraded in the endocytic pathway as efficiently as wildtype PA by most normal cell types due to the limited expression of MMPs suggested that antigen presenting cells (such as dendritic cells and macrophages) may not efficiently present PA-L1 peptides via MHC class II pathway to induce humoral immune response. To test this possibility, we administered (i.p.) 6 doses of PA or PA-L1 into C57BL/6 mice using the same schedule as in the tumor treatment studies. Ten days later the mice were bled, and the PA-neutralizing antibody activities measured. Significantly, we found that the PA-neutralizing antibody titers from wild-type PA treated mice were much higher (−6 fold) than those treated with PA-L1(FIG. 2K). These results indicated that the MMP-activated toxin has much lower immunogenicity compared to the wild-type toxin, suggesting that the engineered toxin might be used for several cycles of treatment without compromising its therapeutic activity.

### Example 5

### The Potent Anti-Tumor Activity of the MMP-Activated Anthrax Lethal Toxin is Not Solely Dependent on its Inhibitory Effect on IL8

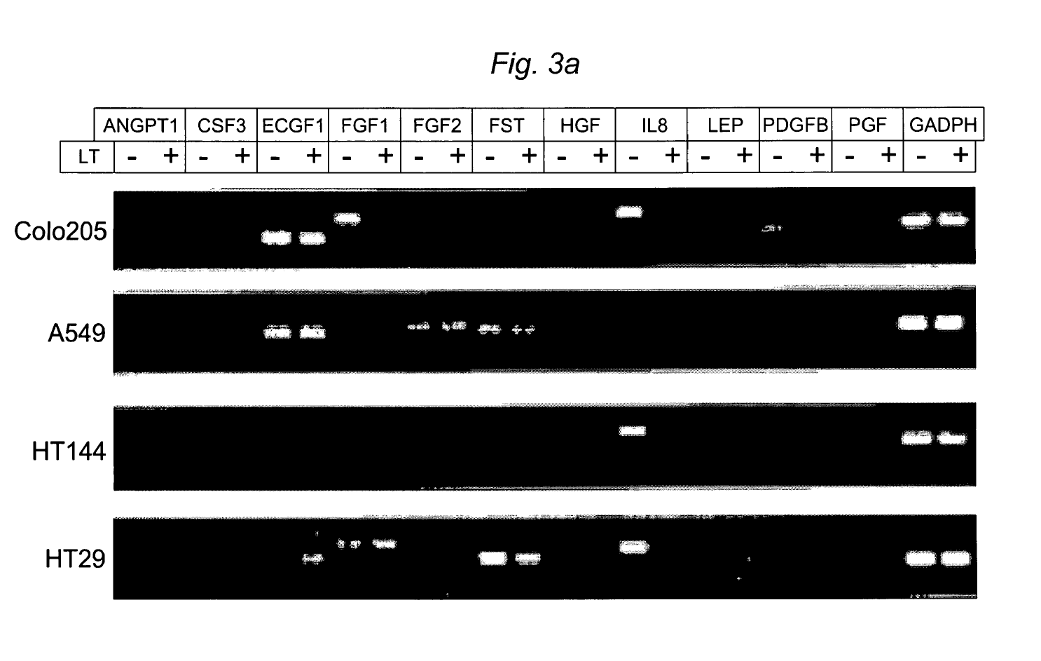

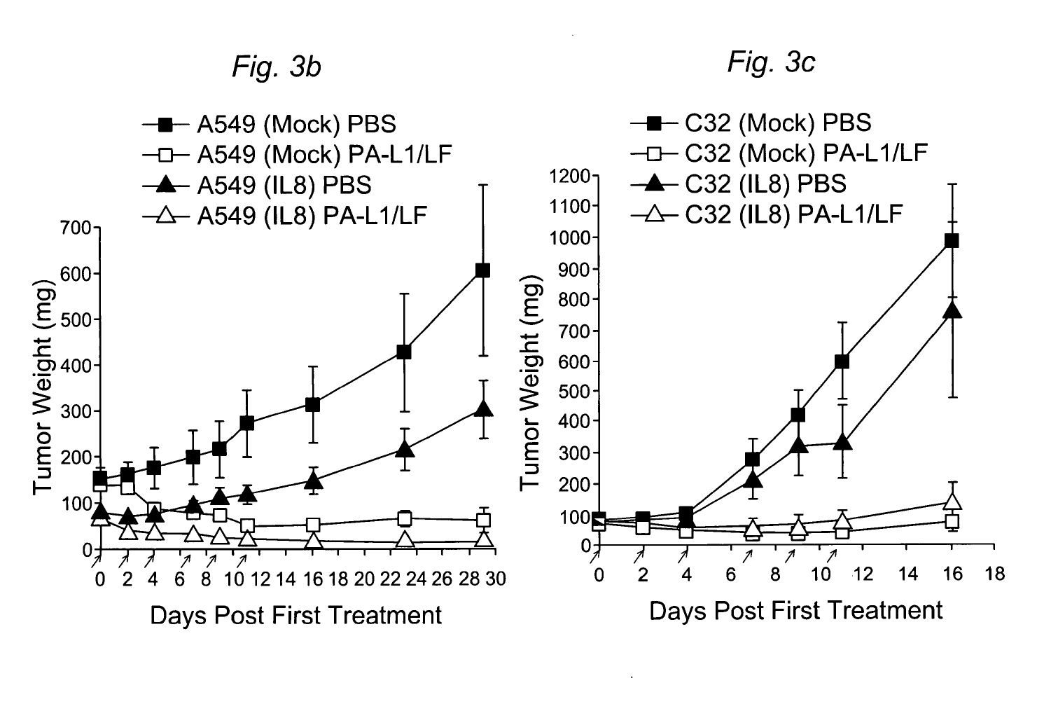

In tumor tissues, cancer cells usually induce tumor angiogenesis by communicating with tumor stromal cells (such as fibroblasts, macrophages, endothelial cells, etc.) by either direct interactions or through secretion of various growth factors and angiogenic factors (Sparmann, A. and Bar-Sagi, D., Cancer Cell, 6:447-458 (2004); Mizukami, Y. et al., Nat. Med., 11:992-997 (2005); Zeng, Q. et al., Cancer Cell, 8:13-23 (2005)). To determine whether LT can affect expression of angiogenic factors by cancer cells, we performed human angiogenic factor profiling analyses with four human cancer cells A549/ATCC, HT144, Colo205, and HT29 cells using the MultiGene-12 RT-PCR Profiling Kit (SuperArray Bioscience Corporation). The effects of LT treatment on expression of 11 well-characterized angiogenic factors were evaluated using these cancer cells (FIG. 3A). We showed that interleukin-8 (IL8) was the only factor down-regulated by LT treatment in all four cell lines (FIG. 3A). Further analysis revealed that the expression of vascular endothelial growth factor (VEGF) by these cancer cells was not affected by LT treatment (data not shown). These findings, together with the results from a previous study showing that LT can down-regulate IL8 expression in human umbilical endothelial cells (HUVEC) (Batty et al., 2006), suggest that many cell types may share a common LT-susceptible pathway for regulating IL8.

It is well established that IL8 plays an important role in tumor angiogenesis, and that IL8 has been demonstrated as an effective target in tumor therapy in animal models (Sparmann, A. and Bar-Sagi, D., Cancer Cell, 6:447-458 (2004); Mizukami, Y. et al., Nat. Med., 11:992-997 (2005)). We therefore asked whether the inhibitory effect of LT on IL8 could account for the potent anti-tumor activity of PA-L1/LF. To do so, we cloned a human IL8 cDNA fragment lacking the 3′ untranslated region which contains an AU-rich element through which LT regulates IL8 mRNA stability (Batty, S. et al., Cell Microbiol., 8:130-138 (2006)). This LT ‘resistant’ IL8 coding sequence was subcloned into a mammalian expression vector, pIRESHgy2b, under the control of the CMV promoter, and transfected into A549/ATCC and C32 cells. Stable cell clones expressing the exogenous IL8 were isolated and expression of the exogenous IL8 was confirmed to be unaffected by PA/LF treatment (data not shown). These IL8-transfected cells and the empty vector-transfected cells were pooled separately, and used to establish tumor xenografts in nude mice. The tumor-bearing mice were treated with 6 doses of PBS or 30/10 μg of PA-L1/LF. The results showed that the strong anti-tumor efficacy of PA-L1/LF was not compromised in either A549/ATCC or C32 tumors with “resistant” IL8 (FIG. 3B and FIG. 3C). These results demonstrate that the potent anti-tumor activity of PA-L1/LF is not solely dependent on its inhibitory effect on IL8. In both cases, we observed that the tumors over-expressing IL8 grew slower than the tumors transfected with the empty vector (FIG. 3B and FIG. 3C). The reason for this phenomenon is unclear; one possibility is that the over-expressed IL8 may trigger innate immune responses due to its chemotactic activities for neutrophils and macrophages, providing an unfavorable microenvironment for tumor growth.

### Example 6

### MMP-Activated Anthrax Lethal Toxin Demonstrates Potent Anti-Angiogenic Activity

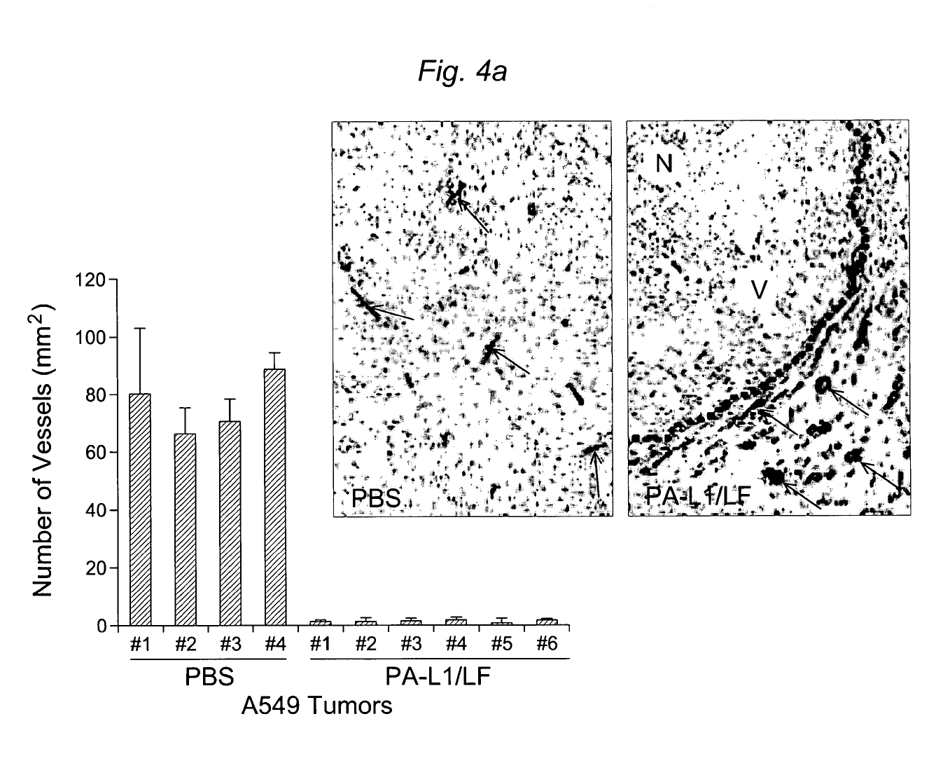

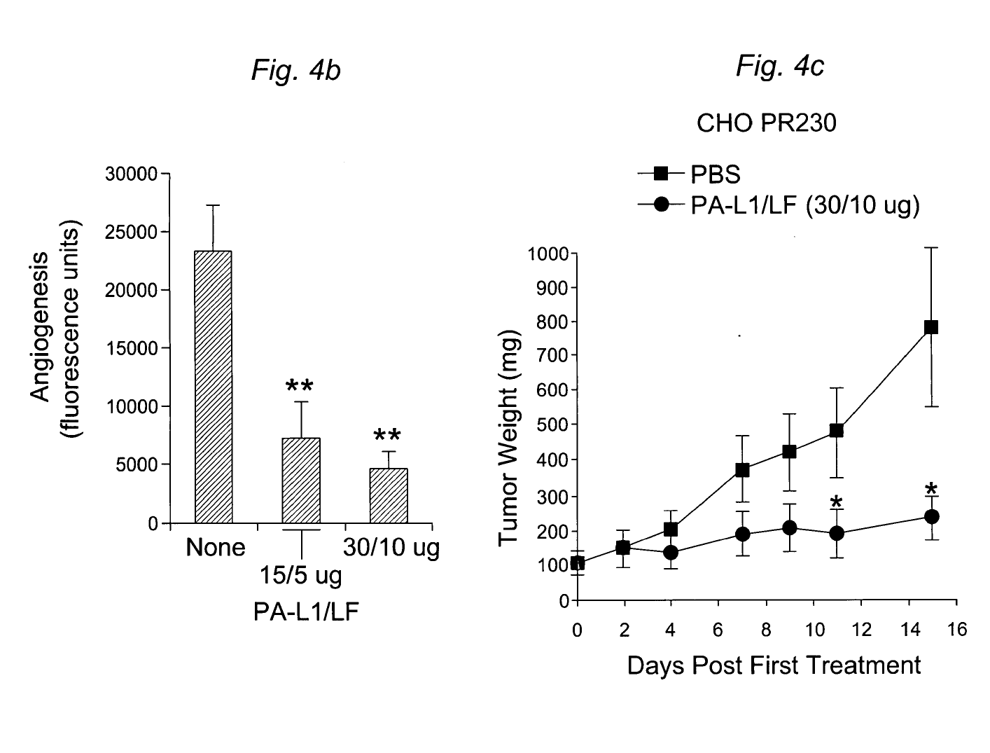

We next attempted to determine the underlying mechanism of the potent anti-tumor activity of systemic administration of PA-L1/LF. To investigate the effects of PA-L1/LF on tumor vasculature and angiogenesis, we stained A549/ATCC tumors isolated from mice treated with either PBS or PA-L1/LF using an antibody against the endothelial cell surface marker CD31. Notably, microvascular structures were easily detected in the PBS-treated tumors, but hardly detected in the toxin-treated tumors, even within the viable tumor areas (FIG. 4A). Importantly, the endothelial cells in the normal surrounding tissues of the toxin-treated tumors remained intact (FIG. 4A, insets), suggesting that the anti-vasculature and -angiogenic activity of PA-L1/LF is tumor-specific. This is likely due to the fact that the endothelial cells in normal tissues are relatively quiescent and lack expression of MMPs, and therefore MEK-independent, whereas those in tumor tissues enriched with angiogenic factors and growth factors are highly proliferative, express MMPs, and are MEK-dependent.

To more directly evaluate the effect of PA-L1/LF on angiogenesis in vivo, we performed the directed in vivo angiogenesis assay (DIVAA) (Guedez, L. et al., Am. J Pathol., 162:1431-1439 (2003)) by subcutaneously implanting nude mice with “angioreactors” containing basement membrane extracts, VEGF, and FGF2. Then the mice were treated (i.p.) with 6 doses of PBS or PA-L1/LF. Significantly, both the 15/5 μg and 30/10 μg doses of PA-L1/LF efficiently decreased in vivo angiogenesis (FIG. 4B). These results, together with those described above, suggested that the potent and broad anti-tumor activity of the MMP-activated LT is due largely to the indirect targeting of tumor vasculature and angiogenic processes.

To directly test this hypothesis, we next used tumor cells that were rendered deficient in anthrax toxin receptors (Liu, S. and Leppla, S. H., Mol. Cell, 12:603-613 (2003)). Thus, the anthrax toxin receptors-deficient Chinese hamster ovary (CHO) cell line, PR230, which cannot bind PA proteins (Liu, S. and Leppla, S. H., Mol. Cell, 12:603-613 (2003)) was xenografted to mice, and the mice were treated with PA-L1/LF or PBS. Consistent with our hypothesis, anthrax toxin receptor-ablated CHO cells remained highly sensitive to PA-L1/LF treatment (FIG. 4C).

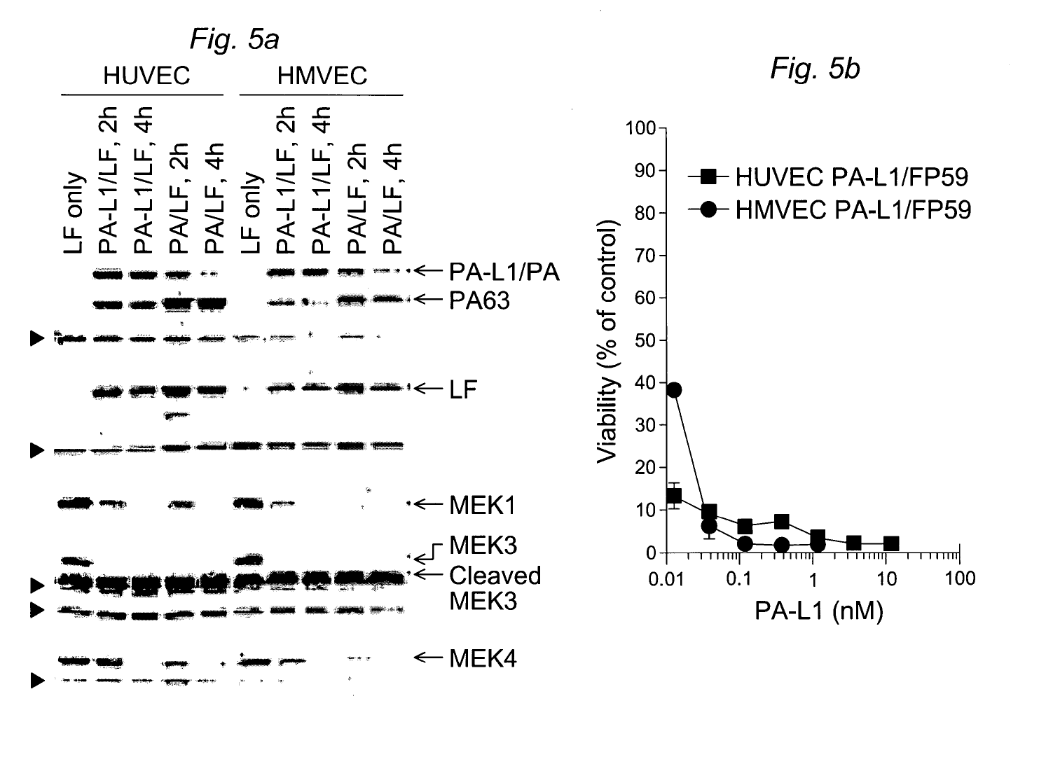

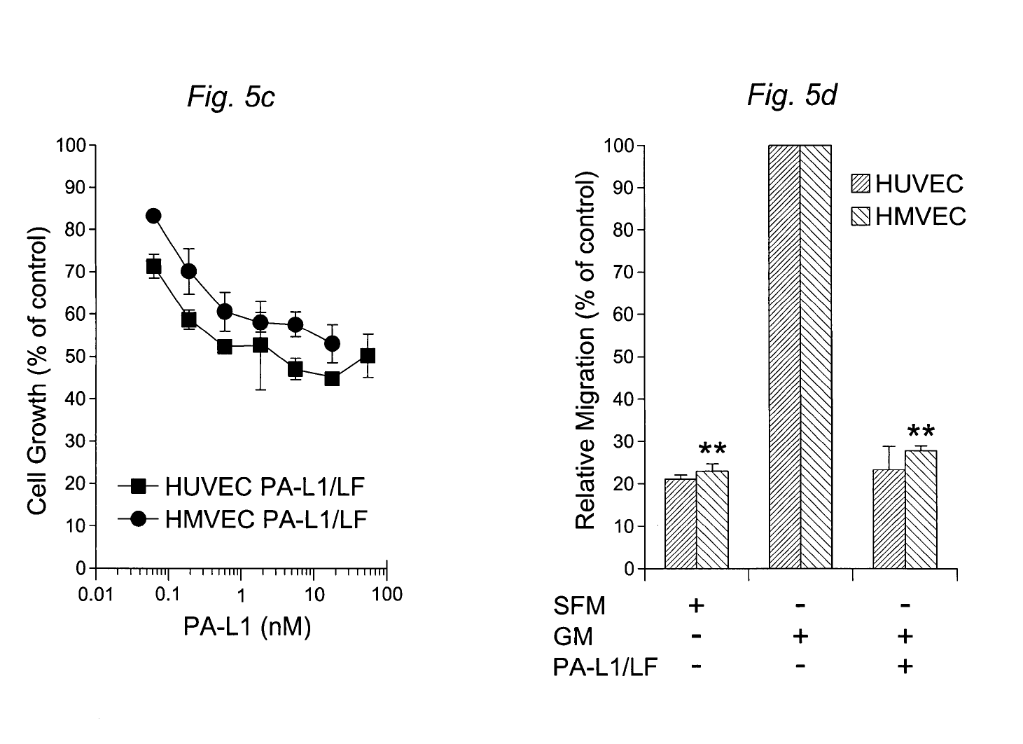

To investigate whether the functions of endothelial cells could be directly impacted by PA-L1/LF, two human primary endothelial cells, HMVEC (human microvascular endothelial cells) and HUVEC, were used for further analysis. As expected, these cells could efficiently bind and proteolytically process PA or PA-L1 to the active PA63 form, demonstrating that these two highly proliferating endothelial cells cultured in growth factor- and angiogenic factor-enriched medium (mimicking tumor environments) express furin as well as MMP activities. Further, these primary endothelial cells could bind and translocate LF into the cytosol of the cells, resulting in MEK1, MEK3, and MEK4 cleavage in a PA protein-dependent manner (FIG. 5A). In agreement with the result that these cells express MMP activities in test culture conditions, these endothelial cells were highly sensitive to PA-L1/FP59 (FIG. 5B). Moreover, the growth of these cells was modestly inhibited by PA-L1/LF, with 50% inhibition observed after 72 h incubation with toxin (FIG. 5C). Of note, migration of both these endothelial cells toward a gradient of serum and angiogenic factors (FGFb and VEGF) was significantly perturbed (FIG. 5D). These results are consistent with the notion that PA-L1/LF can inhibit endothelial cell proliferation and migration, which play a critical role in tumor angiogenesis.

### Example 7

### MMP-Activated Anthrax Lethal Toxin Delays, but does not Abrogate, Skin Wound Healing

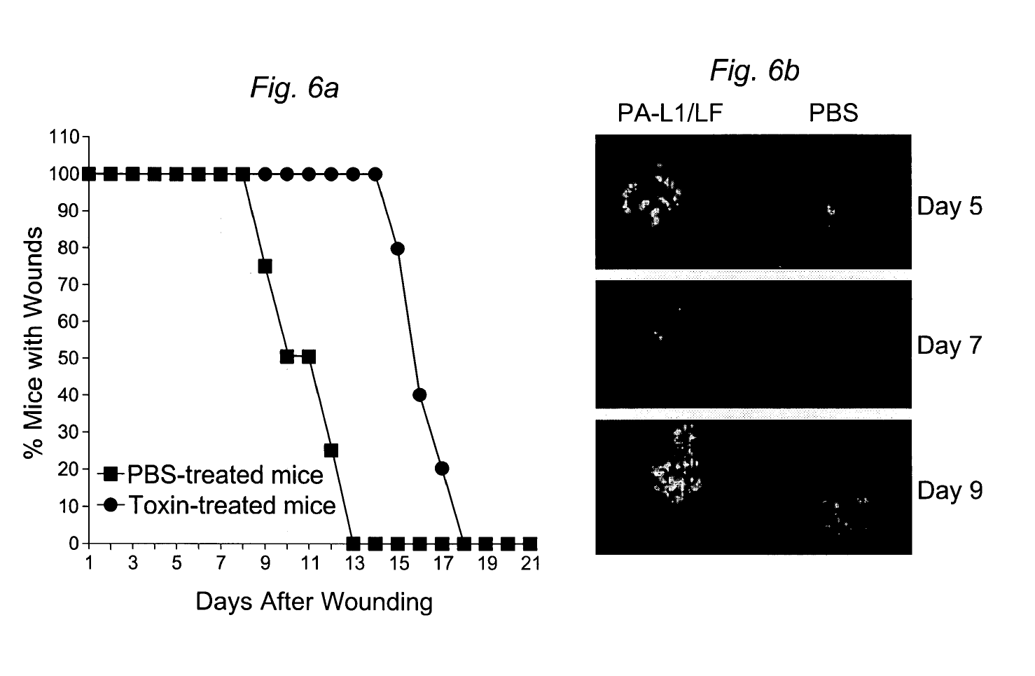

Many post-developmental tissue repair and tissue remodeling processes are dependent on angiogenesis. Furthermore, tumor angiogenesis is believed to recapitulate important aspects of physiological angiogenesis (Dvorak, H. F., N. Engl. J. Med., 315:1650-1659 (1986)). Skin wound healing is one such physiological tissue remodeling process that is associated with extensive neo-angiogenesis (Singer, A. J. and Clark, R. A., N. Engl. J. Med., 341:738-746 (1999)). Thus, the above results predict that PA-L1/LF may also affect the skin wound healing process, potentially complicating the clinical use of PA-L1/LF. To test the effects of PA-L1/LF on physiological angiogenesis, full-thickness incisional skin wounds were made in C57BL/6 mice. The mice were then treated (three times per week) with either PA-L1/LF (30/10 ug) or PBS, and the wound healing time was determined (FIG. 6). No overt qualitative macroscopic differences were observed in healing wounds from toxin-treated and mock-treated mice (FIG. 6B). However, toxin-treated mice displayed a fifty percent delay in the average healing time, showing that systemic PA-L1/LF treatment moderately impairs, but does not abrogate, a physiological tissue repair process (FIG. 6).

### Example 8

### Experimental Procedures

### Protein Purification

PA, PA-L1, LF, and FP59 were purified as previously described (Liu, S. et al., Cell. Microb. (2006)).

### Cell Culture and Cytotoxicity Assay

All NCI60 human cancer cells and mouse melanoma B16-BL6 and Lewis lung carcinoma LL3 cells were cultured in DMEM with 10% fetal bovine serum (FBS) as described previously (Liu, S. et al., J. Biol. Chem., 276:17976-17984 (2001); Liu, S, and Leppla, S. H., Mol. Cell, 12:603-613 (2003)). Human primary endothelial cells HMVEC and HMVEC were obtained from Cambrex (Walkersville, Md.) HMVEC and HMVEC were cultured in endothelial cell growth medium-2 (EGM-2) plus EGM-2 singleQuots and EGM-2 plus EGM-2 MV singleQuots (Cambrex), respectively. Mouse bone marrow derived macrophages were isolated from C57BL/6, BALB/c, and nude mice as described (Swanson, M. S. and Isberg, R. R., Infect. Itrmiun., 63:3609-3620 (1995)). For cytotoxicity assays, approximately 5,000 cells were seeded into each well in 96-well plates. Then various concentrations of PA proteins, combined with LF (5.5 nM) or FP59 (1.9 nM), were added to the cells. Cell viability was assayed after incubation with the toxins for 72 h using MTT (3[4,5-dimethylthiazol-2-yl]-2,5-diphenyltetrazolium bromide), as described previously Liu, S. et al., Cancer Res., 60:6061-6067 (2000)).

### PA Proteins Binding, LF Translocation, and MEKs Cleavage Analyses

HUVEC and HMVEC cells grown to confluence in 6-well plates were incubated with growth medium containing PA/LF (6 nM/6 nM) or PA-L1/LF (6 nM/6 nM) for 2 h or 4 h at 37° C., then washed five times with Hank's Balanced Salt Solution (HBSS) (Biofluids, Rockville, Md.) to remove unbound toxins. The cells were then lysed and the cell lysates were subjected to SDS-PAGE, followed by Western blotting to detect cell-associated PA proteins, LF, and MEKs cleavages. Anti-PA polyclonal rabbit antiserum (#5308) and anti-LF antiserum (#5309) were made in our laboratory. Anti-MEK1 (Cat No. 07-641) was obtained from Upstate Biotechnology, Inc. (Lake Placid, N.Y.), anti-MEK3 (Cat No. sc-961) and anti-MEK4 (Cat No. sc-837) from Santa Cruz Biotechnology, Inc. (Santa Cruz, Calif.).

Maximum Tolerated Dose Determination

Female C57BL/6J and BALB/c mice (The Jackson Laboratory) between 8-10 weeks of age were used in this study. The mice were housed in a pathogen-free facility certified by the Association for Assessment and Accreditation of Laboratory Animal Care International, and the study was carried out in accordance with NIH guidelines. The maximum tolerated doses of PA/LF (3:1 ratio) and PA-L1/LF (3:1 ratio) were determined using a dose escalation protocol aimed at minimizing the number of the mice used. The mice (n=5) in each group were anesthetized by isoflurane inhalation and injected intraperitoneally with 6 doses of the toxins in 500 μl PBS using the schedule of three times a week for two weeks. The mice were monitored closely for signs of toxicity including inactivity, loss of appetite, inability to groom, ruffling of fur, and shortness of breath, and euthanized by CO₂ inhalation at the onset of obvious malaise. The maximum tolerated dose for 6 administrations (MTD6) was determined as the highest dose in which outward disease was not observed in any mice within a 14-day period of observation.

### Histopathological Analysis

To evaluate the in vivo toxicity of the lethal toxins, C57BL/6 mice were injected with 6 doses of PBS and 45/15 μg of PA-L1/LF. Then the mice were killed by a brief CO₂ inhalation. The organs and tissues, including brain, lung, heart, liver, small and large intestines, kidney and adrenal glands, stomach, pancreas, spleen, thyroid, bladder, esophagus, skeletal muscles, thymus, and lymph nodes were fixed for 24 h in 4% paraformaldehyde, embedded in paraffin, sectioned, and stained with hematoxylin/eosin and subjected to microscopic analysis.

### In Vivo Anti-Tumor Experiments

Various human tumor xenografts were established in nude mice (NCI, Frederick, Md.) by subcutaneously injecting 1×10⁷ human tumor cells into the dorsal region of each mouse. The syngeneic mouse B16-BL6 melanoma and LU Lewis lung carcinoma were established subcutaneously in C57BL/6 mice by injecting 5×10⁵ cells per mouse. After the human tumor xenografts were well established and the mouse transplanted tumors were visible, the tumor-bearing mice were injected (i.p.) with PA/LF, PA-L1/LF, or PBS in 500 ul PBS for 6 doses (three times per week for two weeks). The longest and shortest tumor diameters were determined with calipers by an investigator unaware of the treatment group, and the tumor weight was calculated using the formula: milligrams=(length in mm×[width in mm]²)/2. The experiment was terminated when one or more mice in a treatment group presented frank tumor ulceration or the tumor exceeded 10% of body weight. The significance of differences in tumor size was determined by two-tailed Student's t-test using Microsoft Excel.

### Tumor Histology and Immunohistochemistry

A549/ATCC tumor-bearing nude mice were treated (i.p.) with 30/10 μg of PA-L1/LF or PBS at day 0, 2, 4, and 7. The mice were euthanized 2 h after BrdU injection (i.p.) at day 8. The tumors were dissected and fixed for 24 h in 4% paraformaldehyde, embedded in paraffin, sectioned, and stained with hematoxylin/eosin. The tumor sections were also analyzed using a monoclonal rat anti-mouse CD31 (Research Diagnostics Inc, Concord, Mass.), or a monoclonal rat anti-human BrdU (Accurate Chemical & Scientific Corporation, Westbury, N.Y.). Images of the histological sections were captured using an Aperio T3 Scanscope (Aperio Technologies, Vista, Calif.), saved as TIFF files, and were quantified using the Northern Eclipse Image Analysis Software (Empix Imaging, North Tonawanda, N.Y.). For necrosis, the results were expressed as a percentage of necrotic area to total area. Cell proliferation is presented as a percentage of BrdU positive cells among total cells. Tumor vascularization is shown as the number of CD31-positive structures per min². All histological evaluation was performed by an investigator that was blinded as to the treatment of each mouse.

### Angiogenic Factors Profiling Reverse Transcription (RT)-PCR Analysis

Human cancer A549/ATCC, HT144, HT29, SK-MEL-28 cells were cultured into 6-well plates to 50% confluence and treated with DMEM only or DMEM containing PA-L1/LF (2.4/2.2 nM) overnight. Total RNA was then isolated and subjected for the first-strand cDNA synthesis using the SuperScript II Reverse Transcriptase (Invitrogen). Then, the RT products were used as the templates for the angiogenic factor profiling PCR analysis using the kit purchased from SuperArray Bioscience (PH-065B) (Frederick, Md.) following the manufacturer's instructions.

### RT-PCR and Transfection

Total RNA isolated from human A594/ATCC cells was subjected to the reverse transcription reaction using the SuperScript II Reverse Transcriptase (Invitrogen). The human IL8 cDNA coding fragment was then amplified using a forward primer AATTCTTAAGCCACCATGACTTCCAAGCTGGCCGTGGCTCTCTT (AflII site is underlined, Kozak sequence in italic, start codon in boldface) and a reverse primer GGAGGATCCTTATGAATTCTCAGCCCTCTTCAAAAACT (BamHI site underlined). The resulting DNA fragment was subcloned into AflII and BamHI sites of pIREShgy2B, a bicistronic mammalian expression vector containing an attenuated version of the internal ribosome entry site of the encephalomyocarditis virus, which allows both the gene of interest and the hygromycin B selection marker to be translated from a single mRNA. The resulting IL8 expression plasmid (confirmed by DNA sequencing) and the empty control vector were transfected into A549/ATCC or C32 cells using Lipofectamine 2000 reagent (Invitrogen). Stably transfected cells were selected by growing them in hygromycin B (500 μg/ml) for two weeks. The colonies expressing the exogenous IL8 were confirmed by RT-PCR using a forward IL8 primer paired with a reverse vector-specific primer. The clones expressing the exogenous IL8 or transfected with an empty vector were pooled separately and used for establishment of tumor xenografts to test their response to PA-L1/LF.

### Cell Migration Assay

A CytoSelect 24-well cell migration assay kit (Cat. CBA-100-C) purchased from Cell Biolabs (San Diego, Calif.) was used for the assay. HUVEC and HMVEC cells pretreated with or without PA-L1/LF (2.4 nM/2.2 nM) for 2 h, were trypsinized and re-suspended in EGM2 (without MV singleQuots) with or without the same concentration of PA-L1/LF at a density of 1×10⁶ cells/ml.

The cells were added into the cell culture inserts (300 ul/well), which were then placed into a 24-well plate containing EGM-2 only or EGM-2 plus MV singleQuots (the complete growth medium containing 5% FBS and angiogenic and growth factors VEGF, FGF2, EGF, and IGF), and incubated for 16 h. Cells which migrated to the other sides of the inserts were stained and measured following the manufacturer's instructions.

### In Vivo Angiogenesis Assay

DIVAA was performed using a DIVAA Starter Kit (Trevigen, Gaithersburg, Md.) following the kit manual. Anesthetized 8-week nude mice (NCI, Frederick) were subcutaneously implanted with Trevigen's basement membrane extract and VEGF and FGF2-containing angioreactors under sterile surgical conditions (day 0). Then the mice were treated with 6 doses of PA-L1/LF or PBS at day 3, 5, 7, 10, 12, and 14. The mice were euthanized by CO₂ inhalation at day 16, and the angioreactors were removed. The vascular endothelial cells which had grown into the reactors were quantitated according to the manufacturer's instructions.

### Wound Healing Experiment

Skin wound healing was performed essentially as described (Bugge, T. H. et al., Cell, 87:709-719 (1996)). Briefly, C57BL/6.1 mice (8-10 weeks) were randomly divided into two groups and anesthetized by inhalation of 2% isoflurane before surgical incision. Fifteen mm long full-thickness incisional wounds were made in the shaved middorsal skin. The wounds were neither dressed nor sutured. Starting immediately after wounding, one group was treated with PA-L1/LF (30/10 μg) and the second group was treated with PBS three times per week until all the wounds were healed. The rate of wound healing was determined by daily inspection and the wound was scored as healed when only a minimal residual skin defect was apparent. Surgery and evaluation of the macroscopic progress of wound healing was done by an investigator that was blinded as to the treatment of the mice.

All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A method of inhibiting tumor associated angiogenesis in a subject, the method comprising the steps of:
(1) administering to the subject a therapeutically effective amount of a mutant PA protein comprising a matrix metalloproteinase 2-recognized cleavage site in place of the native PA furin-recognized cleavage site, wherein the mutant PA is cleaved by a matrix metalloproteinase; and
(ii) administering to the subject a therapeutically effective amount of an LF polypeptide comprising a PA binding site; wherein the LF polypeptide binds to cleaved PA and is translocated into a tumor associated endothelial cell, thereby inhibiting tumor angiogenesis.

2. The method of claim 1, said tumor is a solid tumor.

3. The method of claim 2, wherein said solid tumor is selected from the group consisting of lung cancer, colon cancer, melanoma, breast cancer, bladder cancer, thyroid cancer, liver cancer, pleural cancer, pancreatic cancer, ovarian cancer, cervical cancer, fibrosarcoma, neuroblastoma, and glioma.

4. The method of claim 2, wherein said solid tumor is selected from the group consisting of lung cancer, colon cancer, and melanoma.

5. The method of claim 1, wherein the LF polypeptide is native LF.

6. The method of claim 1, wherein the LF polypeptide is LFn.

7. The method of claim 1, wherein the LF polypeptide is a fusion protein.

8. The method of claim 1, wherein the mutant PA protein and the LF polypeptide are administered systemically to the subject.

9. The method of claim 1, wherein said matrix metalloproteinase 2 cleavage site has the sequence GPLGMLSQ.

10. The method of claim 1, wherein said mutant PA is cleaved by a matrix metalloproteinase 2 from endothelial cells.

11. The method of claim 1, wherein said PA and LF, after translocation into a tumor associated endothelial cell, induces apoptosis of said endothelial cell.

12. The method of claim 1, wherein said endothelial cell has an activated MAP kinase pathway.

13. The method of claim 1, wherein said translocated LF polypeptide and cleaved PA results in cleavage of a MEK selected from the group consisting of MEK1, MEK2, MEK3, MEK4, MEK6, and MEK7.

14. The method of claim 1, wherein said mutant PA is further cleaved by a matrix metalloproteinase 2 from a tumor cell.

15. The method of claim 14, wherein said LF polypeptide binds to cleaved PA and is translocated into the tumor cell.

16. The method of claim 15, wherein said translocated LF polypeptide and cleaved PA inhibit the expression of IL-8 mRNA in the tumor cell.

17. The method of claim 14, wherein said tumor cell has an activated MAP kinase pathway.

18. The method of claim 17, wherein said activated MAP kinase pathway is due to a BRAF V600E mutation.

19. The method of claim 15, wherein said translocated LF polypeptide and cleaved PA results in cleavage of a MEK selected from the group consisting of MEK1, MEK2, MEK3, MEK4, MEK6, and MEK7.
